(19) United States
(12) Patent Application Publication
(10) Pub. No.: US 2003/0207792 A1
(43) Pub. Date: Nov. 6, 2003

(54) Treating cancer

(76) Inventors: Hilmar Meek Warenius, Heswall (GB); Philip Spencer Rudland, Liverpool (GB); Laurence Anthony Seabra, Heswall (GB)

Correspondence Address: ROTHWELL, FIGG, ERNST & MANBECK, P.C., 1425 K STREET, N.W., SUITE 800, WASHINGTON, DC, 20005, US

(21) Appl. No.: 10/272,301

(22) Filed: Oct. 17, 2002

(30) Foreign Application Priority Data
Mar. 25, 2002 (GB) 0207031.6
Jul. 17, 2002 (GB) 0216634.6

(51) Int. Cl. A61K 31/00; C12Q 1/68

(52) U.S. Cl. 514/1; 435/6

(57) ABSTRACT

Provided is a method for screening for effective agents for the treatment of cancer which method comprises testing a putative agent that is likely to disrupt a critical normal gene product present in said cancer for its cytotoxic and/or growth inhibitory effect upon a cancer cell sample, and identifying an effective agents as an agent which is cytotoxic to, and/or inhibiting to the growth of the cancer cell sample. This invention further provides an agent capable of disrupting a critical normal gene in such a manner as to be cytotoxic to, or inhibiting to the growth of a cancer cell, and a method of treating a cancer patient with such an agent.

The present application concerns a method of selecting an effective agent for the treatment of cancer, including metastatic cancer. The application is particularly concerned with the identification of suitable targets for anti-cancer agents. More particularly, the application identifies a region of the CDK4 gene product responsible for a critical normal function of the gene product as a suitable target for anti-cancer agents.

Although chemotherapy has been responsible for curing many people of cancer in the latter half of the 20th century, there still remain a large number of patients whose tumours either show little response to treatment, or respond initially only to recur later. For these patients, the current treatments are clearly inadequate.

The majority of the deaths arising from cancers of solid tissues can be ascribed to the process of metastasis wherein cancer cells spread from the site of origin to distant sites in the body. For example, in breast cancer, cancer cells break off from the primary tumour and metastasise via lymphatic and blood vessels. The initial spread of cancer cells is usually to the local lymph nodes, most frequently to the adjacent axilla. Thereafter breast cancer cells can disseminate via the blood stream resulting in distant metastases. These distant metastases are usually the cause of death in the majority of breast cancer patients: Therefore, agents that prevent metastasis would be highly desirable.

There has been substantial investment in researching the mechanisms used by normal eukaryotic cells to control progress through the cell cycle in the hope that this would lead to an understanding of how cancer arises and suggest possible targets for cancer therapy. It is currently understood that progress through the phases of the cell cycle is controlled by a class of enzymes termed “Cyclin Dependent Kinases” (CDKs). Cyclin dependent kinases are serine/threonine cyclin-dependent kinases that are synthesised continuously and maintain relatively constant levels in the cell. They are inactive on their own. CDKs are activated upon binding to their cyclin partner (De Bondt et al. (1993) Nature, 363: 595-602; Jeffrey et al. (1995) Nature, 376: 313-320), and upon phosphorylation by a CAK (cyclin activating kinase; Grana and Reddy (1995) Oncogene, 11: 211-219). CDK kinase activity can be inhibited by removal of the kinase's cyclin partner and by an inhibitory phosphorylation of a tyrosine residue close to the N-terminus of the protein. For example, CDK4 protein is activated by phosphorylation of threonine 164 but inhibited by phosphorylation of tyrosine 17. The activity of a CDK/cyclin holoenzyme can be regulated by cyclin dependent kinase inhibitors (El-Deiry et al. (1993) Cell, 75: 817-825; Harper et dl (1993) Cell, 75: 805-816; Xiong et al. (1993) Nature, 366: 701-704; Polyak et al. (1994), Genes Dev. 8: 9-22; Serrano et al. (1993) Nature 366: 704-707; Serrano et al. (1995) Science, 267: 249-252).

The cell cycle has several checkpoints to ensure that a cell does not replicate its DNA or divide under inappropriate conditions (Hartwell and Weinert (1989) Science, 296: 629-634). Before passing through these checkpoints, a cell must meet certain criteria. Molecular pathways signaling the presence or absence of these criteria influence the decision to cross the checkpoint by affecting activation of the CDK/cyclin holoenzyme responsible for regulating passage through the checkpoint. For example, positive signal transduction pathways relaying signals from cell surface receptors, such as the Ras/Raf/Erk pathway have been demonstrated to influence pRb phosphorylation, through an effect on the cyclin/CDK holoenzyrnes regulating the G1/S checkpoint (Lloyd et al., (1997) Genes Dev. 11: 663-677). Conversely cyclin dependent kinase inhibitors (CDKIs) such as the p16^{INK4}, p27^{KIP1} and p21^{CIP1/WAF1} gene products can arrest cells at the G1/S checkpoint by inhibiting GI cyclin/CDK holoenzymes. The p21^{WAF1/CIP1} gene may be transcriptionally activated by p53 protein providing a mechanism by which p53 protein can arrest normal cells at the G1/S checkpoint (Li et al., (1994) Oncogene, 9: 2261-2268).

Once activated, the CDK/cyclin holoenzyme initiates the events needed for the cell to enter the next phase of the cell cycle. Different CDK/cyclin holoenzymes regulate different checkpoints in the cell cycle (Hunter and Pines (1994) Cell, 79: 573-582; Sherr (1994) Cell 79: 551-555). The initiation of progress from G1 to S phase that occurs when quiescent mammalian cells are stimulated to divide by the presence of growth factors, involves the interaction of the cyclin D family with either the CDK4 gene product or the CDK6 gene product depending on the cell type (Matushime et al. (1994) Mol. Cell Biol. 14: 2066-2076; Mayerson and Harlow (1994) Mol. Cell Biol. 14: 2077-2086). In mammals, other checkpoints are controlled by different CDK/cyclin holoenzymes e.g. late G1/S is regulated by CDK2/cyclin E, progress through S phase by CDK2/cyclin A and late S/G2 by CDK1/cyclin A (Jeffrey et al. (1995) Nature 376: 313-320). Transit from G2 to mitosis is controlled by the CDK1/cyclin B complex in both mammals and yeast (Draetta (1990) Trends Biochem. Sci. 15: 378-382; Murray (1992) Nature, 359: 599-604).

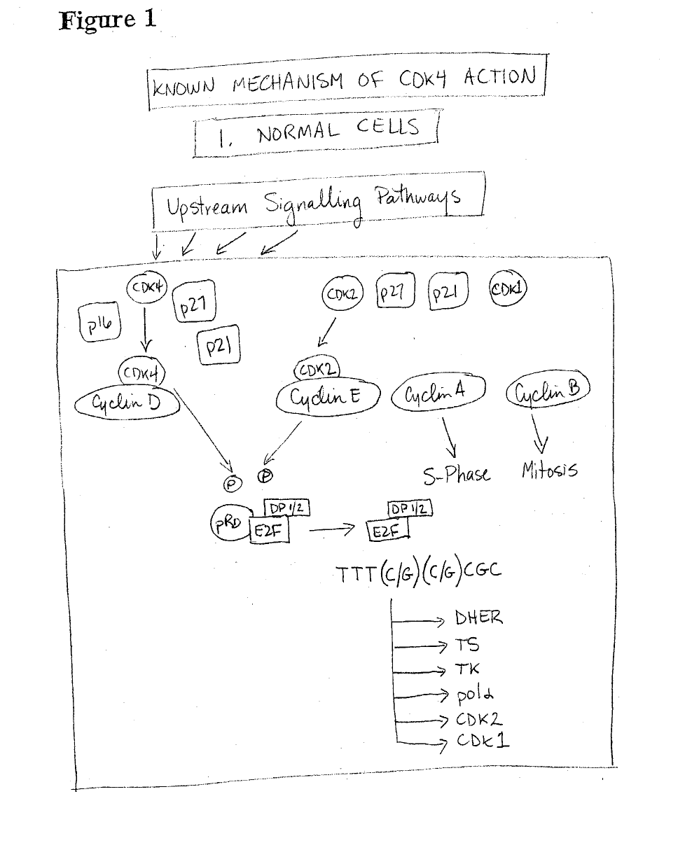

Activation of CDK4 is understood to initiate transit from G1 to S phase. FIG. 1 provides a schematic diagram showing the role of CDK4 at the G1/S transition in normal cells. Activated CDK4 is thought to mediate its effects through phosphorylation of pRb and related proteins p107 and p130. In their hypophosphorylated state pRb, p107 and p130 bind E2F transcription factors. However, upon phosphorylation of pRb, p107 and p130, E2F transcription factors are released (Hijmans et al. (1995) Mol. Cell Biol. 15: 3082-3089). The free E2Fs form heterodimers with the proteins DP-l/DP-2. The E2F/DP heterodimers then bind to DNA and activate transcription of factors required for DNA synthesis (Wu et al. (1995) Mol. Cell Biol. 2536-2546). In addition, free E2F protein upregulates genes controlling cell division such as cyclin E, cyclin A, CDK1 and E2Fs. Overexpression of some members of the E2F family, such as E2F-1, however, does not only promote increased cell division, but can also lead to apoptosis. Adenoviral-mediated transfer of exogenous DNA vectors overexpressing E2F-1 to human colonic adenocarcinoma (Draus et al., (2001), Exp. Mol. Med. 33: 209-219), oesophageal (Yang et al., (2000) Clin. Cancer Res. 6: 1579-1589), melanoma (Dong et al., (1999) Cancer 86: 2021-2033, glioma (Fueyo et al., (1998) Nat. Med. 4: 685-690), breast, ovarian (Hunt et al., (1997) Cancer Res., 57: 4722-4726 and head and neck (Liu et al., (1999) Cancer Gene Ther. 6: 163-171) cancer cell lines induced apoptosis in these cell lines. E2F-1 overexpression caused cells to enter S-phase prematurely and to accumulate in G2/M from which they subsequently exited by undergoing apoptosis.

During carcinogenesis, it is currently thought that normal cells become immortalised as a consequence of disruption of the positive and negative cell signalling pathways and cell cycle control mechanisms described above, for example, amplification and overexpression of cyclins and CDKs. Amplification and overexpression of cyclin D protein occurs in many human tumours (Lammie et al., (1991) Oncogene 6: 439-444, Jiang et al, (1993) Proc. Natl. Acad. Sci USA 90: 9026-9030, Schurring et al, (1992) Oncogene, 7: 355-361, Bartkova et al., (1995) Oncogene, 10: 775-778) and cell lines (Buckley et al., (1993) Oncogene, 8: 2127-2133, Warenius et al., (1996) Int. J. Cancer 67: 224-231. Unscheduled expression of cyclin B1 and cyclin E in inappropriate phases of the cell cycle has also been reported in several leukaemic and solid tumour cell lines (Gong et al, (1994) Cancer Res. 54: 4285-4288). 20-fold amplification of genomic CDK4 DNA with accompanying increases in mRNA expression has been detected in 13.8% of a series of 29 human gliomas (He et al., (1994) Cancer Res. 53: 5535-5541). Similar increases in CDK4 genomic DNA and mRNA have been found in 2 out of 14 human sarcomas (Khatib et al., (1993) Cancer Res. 53: 5535-5541).

Abnormalities in CDK inhibitors particularly mutations and altered expression of p16^{INK4} may also occur (Nobori et al., 1994, Okamoto et al., 1994 Jen et al., 1994). High levels of p16^{INK4} protein have been found to correlate with functional inactivation of the retinoblastoma gene product (Tam et al, (1994) Cancer Res. 54: 5816-5820) whilst overexpression of the CDK4 gene product has been suggested to provide an alternative mechanism to p16^{INK4} gene homozygous deletion.

In summary, it is believed that cancers may arise through an evolutionary process, selecting cells with gene mutations that provide a growth advantage (Ilyas et al. Eur. J. Cancer (1999) 35:335-351). By this means the normal diploid cell is progressively transformed into a fully-fledged cancer cell. Studies of early events in carcinogenesis have revealed several genetic lesions causing errors in the cell division and death pathways (Hanahan and Weinberg, Cell (2000) 100:57-70). Approximately three to seven separate molecular lesions are believed to be required to transform a normal diploid cell into a cancer cell (The Genetic Basis of Human Cancer (1999) Vogelstein and Kinzler, eds.).

Cancer cells may metastasise from the site of origin to distant sites in the body. The protein (Kuukasjarvi et al. Cancer Res. (1997) 57:1597-1604) and mRNA populations (Hashimoto et al. Cancer Res. (1996) 56: 5266-5271) of metastatic and non-metastatic breast cancer cells have been compared in order to identify metastagenes; genes that are differently expressed in metastatic, relative to non-metastatic cells. Metastagenes code for proteins that contribute only to the potentially fatal metastatic spread of cancer cells. They do not contribute to uncontrolled growth or immortalisation. Metastagene products include enzymes such as proteases (Duffy Clin. Exp. Metastasis (1992) 10:145-155), proteins associated with cell adhesion (Iwamura et al. Cancer Res. (1997) 57: 1206-1212) and motility factors (Cajot et al. Cancer Res. (1997) 57: 2593-2597; Meyer-Siegler and Hudson Urology (1996) 48: 448-452). An example of a protein thought to be associated with adhesion is osteopontin (Oates et al. Oncogene (1996) 13: 97-104, Oates et al. Cancer Metastasis Rev. (1997) 17: 1-15; Chen et al, Oncogene (1997) 14: 1581-1588) and one associated with motility is a regulatory calcium ion binding protein, p9ka (S100A4) (Barraclough et al. Eur. J. Biochem (1982) 129: 335-341; Barraclough et al. Nucleic Acids Res. (1984) 21:8097-8114; Ebralidze et al. Genes Dev. (1989) 3: 1086-1093; Gibbs et al. J. Biol. Chem. (1994) 269: 18992-18999).

Based on the present models of cancer and metastasis, attempts have been made at rational drug development. It has been considered that the gene products that are disrupted during carcinogenesis are likely to provide highly specific targets for cancer chemotherapy. Using the targets identified by this approach, new therapeutic agents are now being introduced into the clinic. These include Herceptin, which targets the her/neu cell surface receptor in breast cancer (Sliwkowski et al. (1999) Semin Oncol. 4 suppl.12: 60-70; Baselga Eur J. Cancer (2001) 37suppl.1:18-24), farnesyl transferase inhibitors which target the ras oncogene (Adjei et al. (2000) Cancer Research 60:1871-1877), ONYX015 (an E1B deletion mutant adenovirus), designed to target cancer cells with non-functional TP53 (Nemunaitis et al. (2000) Cancer Research 60:6359-6366), and ST1571, designed to target the translocated abelson kinase in chronic myeloid leukaemia (Mauro and Druker (2001) Oncologist 6:233-238).

Thus, the majority of targets for rational anti-cancer drug development available at present have been defined by studies of early carcinogenesis. However, in contrast to cells studied in early carcinogenesis, profound chromosomal damage can be found in the cell exhibiting the “full malignant phenotype”. The full malignant phenotype is found in clinically advanced tumours. It is characterised by an enormous diversity of structural chromosome damage (The Genetic Basis of Human Cancer (1998) Vogelstein and Kinzler eds.; Mitelman et al. (1997) Nature Genet 15:417-474). In addition, cells having the full malignant phenotype also have widespread changes in gene expression when compared to normal cells (Hough et al. (2000), Cancer Research 60: 6281-6287; Waghray et al (2001) Cancer Res. 61: 4283-4286; Wang et al. (2000) Oncogene 19:1519-1528). Given the number of genetic lesions producing molecular abnormalities within the a cancer cell having the full malignant phenotype, it seems unlikely that the cell can simply be returned to its pre-cancerous, normal diploid phenotype by selectively targeting and inhibiting these abnormal early cancer genes.

There are numerous problems associated with the current approach to rational anti-cancer drug development. These include multiplicity of potential drug targets, tumour heterogeneity and genetic instability.

The number of potential drug targets available to the current approach to rational anti-cancer drug development is large and growing. At present there is no way of telling which of the many abnormal genes and gene products present in a cell having the full malignant phenotype are ultimately likely to prove the most effective drug targets.

The difficulties with rational drug development against selected molecular targets are compounded by tumour heterogeneity. Tumour heterogeneity may result from the random chaotic nature of cell division in clinical tumours. It describes the situation where tumour cells of apparently the same type in different patients, behave differently and show differences in phenotypic expression of gene products including those implicated in the process of carcinogenesis (Shackney and Shankey (1995) Cytometry 21:2-5; Harada et al. (1998) Cancer Research 58-4694-4700). As a result of these differences in the phenotypic expression of gene products, apparently similar tumour cells in different patients may respond differently to a particular anticancer drug with some cells being sensitive whilst others are resistant. This type of drug resistance is called intrinsic resistance.

Even within the same tumour in an individual patient, all cells may not exhibit the same pattern of gene expression so that some cells may be resistant to a certain type of chemotherapy to which others are sensitive. Thus chemotherapy may initially cause tumour shrinkage by killing the sensitive cells but fail to kill the resistant cells. The remaining resistant cells continue dividing to produce a cancer that is no wholly drug resistant. This process is termed acquired resistance.

Therefore, tumour heterogeneity leads to intrinsic resistance and acquired resistance of cancers to new anticancer drugs. This may account for the relatively poor clinical response rates in phase I/II studies of new anticancer agents targeting molecules disrupted in early carcinogenesis.

Genetic instability is found in the majority of cancers, if not all. It allows the possibility of new mutations, occurring throughout the life of a tumour (Genetic Instability in Cancer (1996) Lindahl ed.; Lengauer et al. (1998) Nature 396: 643-649). Certain of these mutations may confer drug resistance to the cells in which they occur. Genetic instability, with its ongoing molecular changes is a further cause of acquired drug resistance and makes human tumours a moving target for the rational design of chemotherapeutic agents.

Therefore, currently available treatments are not always adequate to deal with all cancers. Furthermore, agents directed to gene products involved in early carcinogenesis are unlikely to prevent metastasis.

The present invention aims to overcome the problems associated with the above prior art, and in particular aims to solve the problems of tumour heterogeneity and genetic instability that limit the efficacy of prior art cancer therapies. Accordingly, the present invention provides a method of screening for an agent effective in the treatment of a cancer, which method comprises:

a) selecting a putative agent that is likely to disrupt a critical normal gene product present in said cancer;

b) determining the cytotoxic effect of, and/or the growth inhibiting effect of, the putative agent on a cancer cell sample; and

c) identifying an effective agent as an agent which is cytotoxic to and/or inhibiting to the growth of the cancer cell sample.

Preferably, the cytotoxic effect of, and/or the growth inhibiting effect of the putative agent on a control cell sample is also determined. An effective agent is an agent which is more cytotoxic to, and/or more inhibiting to the growth of the cancer cell sample than the control cell sample.

The invention will now be described in more detail, by way of example only, with reference to the following figures.

FIG. 1 is a schematic diagram showing the known mechanism of action of the CDK4 gene product in normal cells.

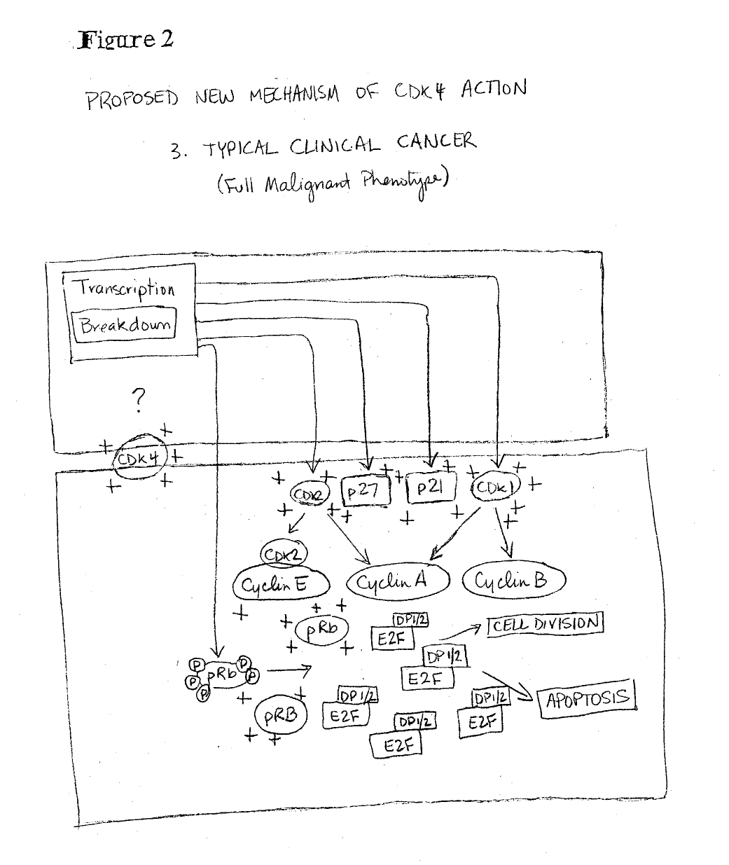

FIG. 2 is a schematic diagram showing a proposed new mechanism of action of the CDK4 gene product in a typical clinical cancer.

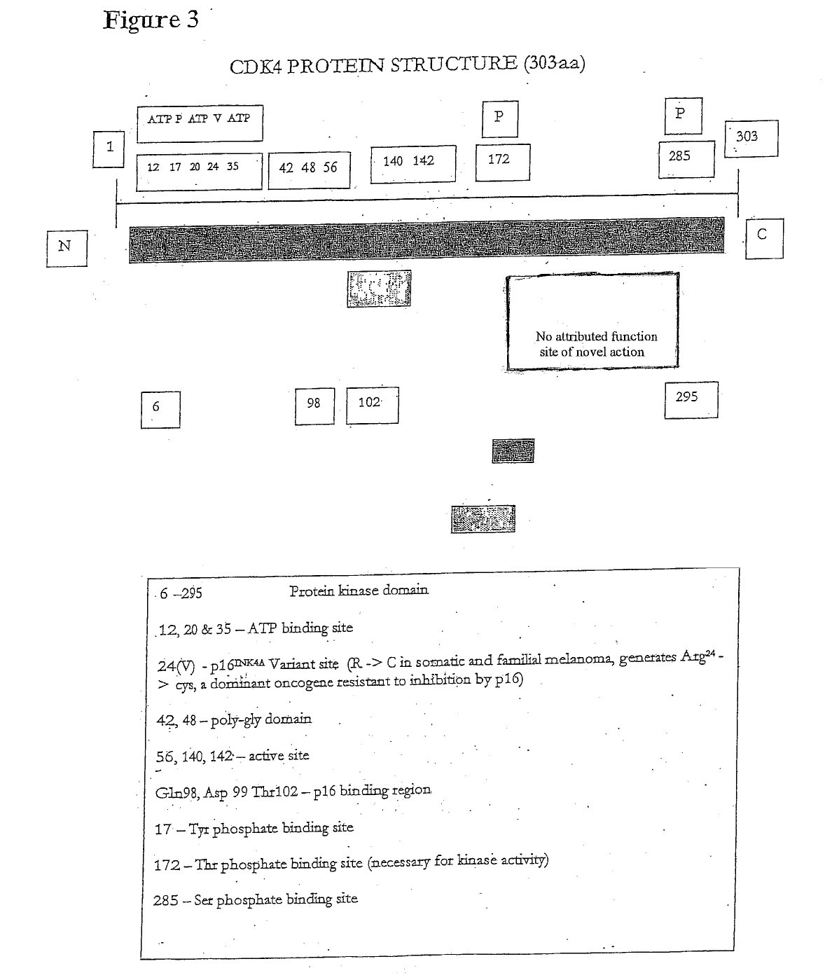

FIG. 3 is a schematic diagram of the CDK4 gene product showing the ATP binding sites, the active site and the sites of phosphorylation. A region between 172 and 285 has no attributed function. This may be the region mediating the novel function of CDK4.

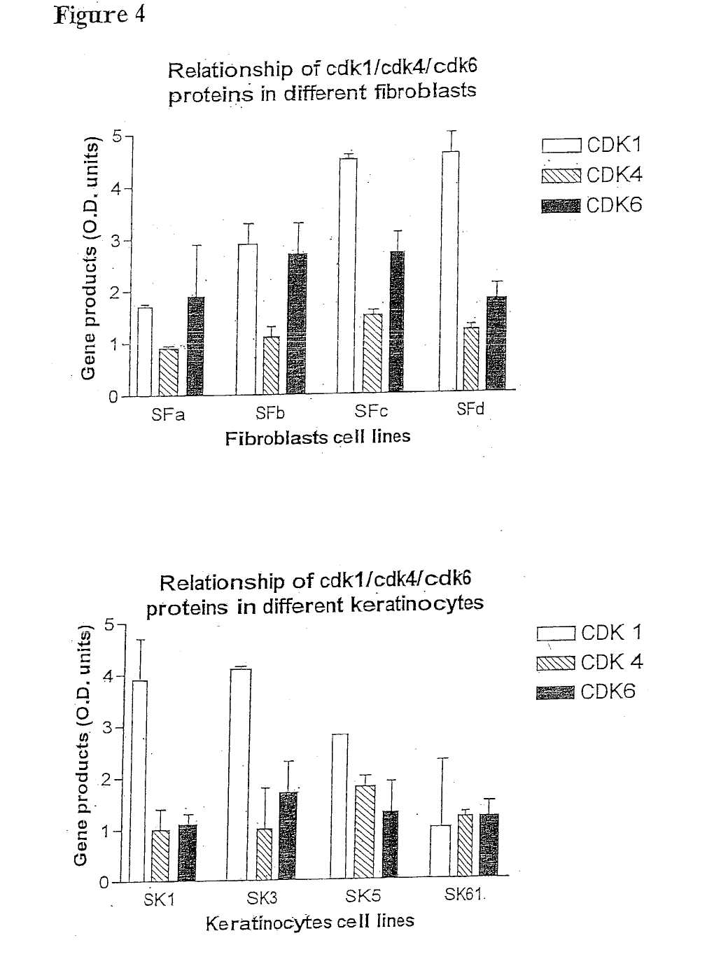

FIG. 4 shows the expression levels of the CDK1, CDK4 and CDK4 gene products in primary cultures of fibroblasts and keratinocyte cell lines. It can be seen that the expression levels of the gene products vary between cell lines, and that the levels of these gene products are not equal within any one cell line.

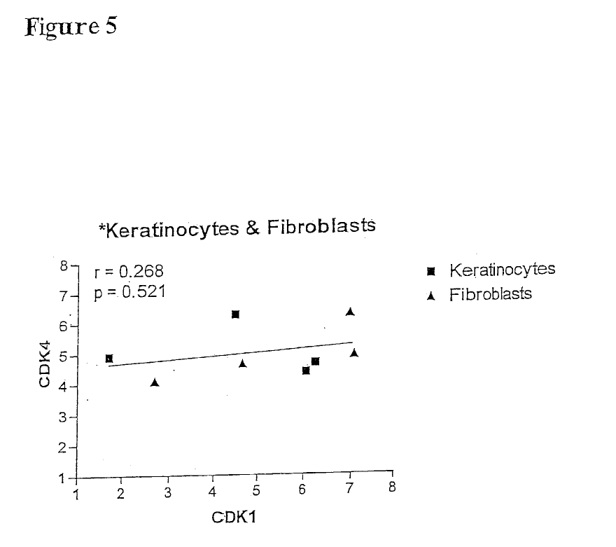

FIG. 5 shows the relationship between the CDK1 and CDK4 gene products in primary cultures of human fibroblast and keratinocyte cell lines. It can be seen that there is no correlation between the levels of these gene products in normal human cells.

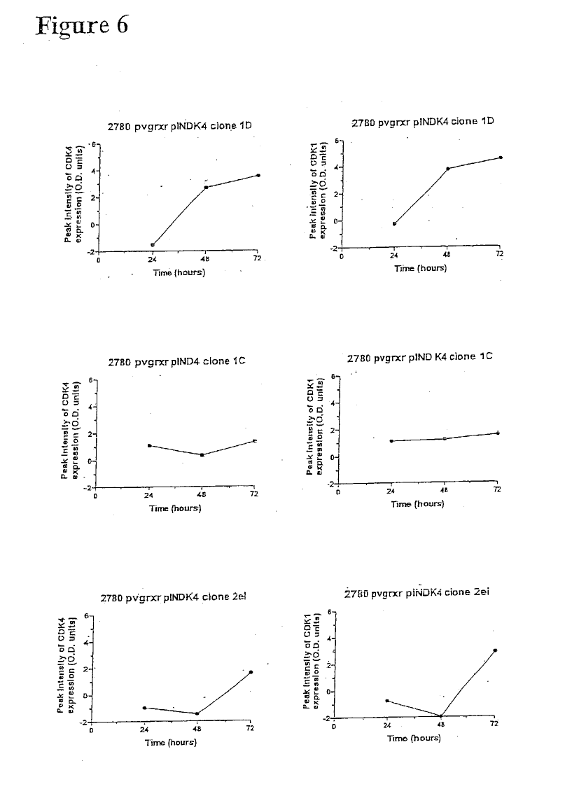

FIG. 6 shows the relationship between the expression level of the CDK4 and CDK1 gene products in a human ovarian cancer cell line. Ovarian cancer cell line 2780 was transfected with pVgRxR and full length wild type CDK4 DNA in pIND. Three transfected clones are depicted: Clone 1D/735 is a high CDK4 expressing transfectant in which marked induction of the CDK4 gene product can be achieved following ponasterone stimulation; clone 2ei is a low-expressing transfectant; and clone 1C shows intermediate expression of the CDK4 gene product on ponasterone induction. Expression of the CDK4 gene product was induced by ponasterone treatment and the levels of the CDK1 and CDK4 gene products at various times after induction were measured by western blotting. It can be seen that the levels of the CDK1 and CDK4 gene products are approximately equal at all times in all clones. In particular, it can be seen that the levels of endogenous cellular CDK1 protein in clone 1D/735 are elevated following ponasterone induction of the expression of exogenous, transfected, CDK4 protein.

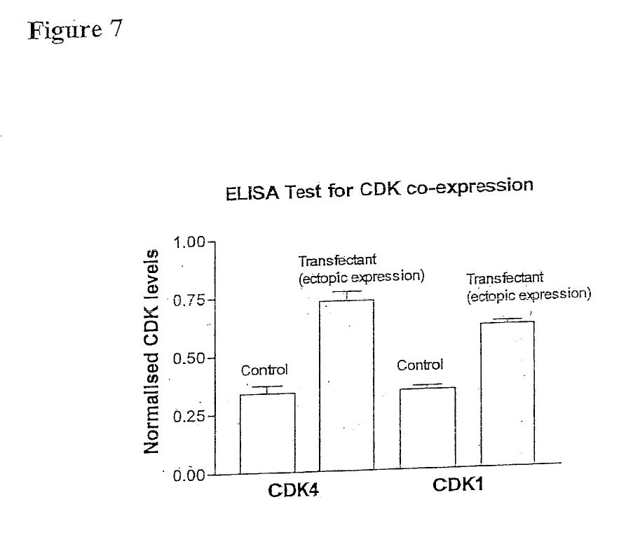

FIG. 7 shows the results of an ELISA assay indicating levels of CDK1 and CDK4 in ponasterone-induced and uninduced clone 2870 1D/735. It can be seen that the levels of both the CDK4 gene product and the CDK1 gene product are elevated 48 h after induction/mock induction.

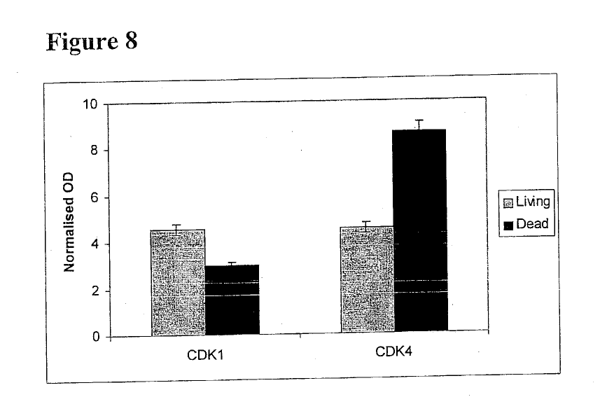

FIG. 8 shows the relationship between the levels of the CDK1 gene product and the CDK4 gene product in proliferating and dying L23COR cells. It can be seen that the ratio of CDK1/CDK4 in proliferating cells is approximately 1.0, while the ratio of CDK1/CDK4 in dying cells is approximately 0.3.

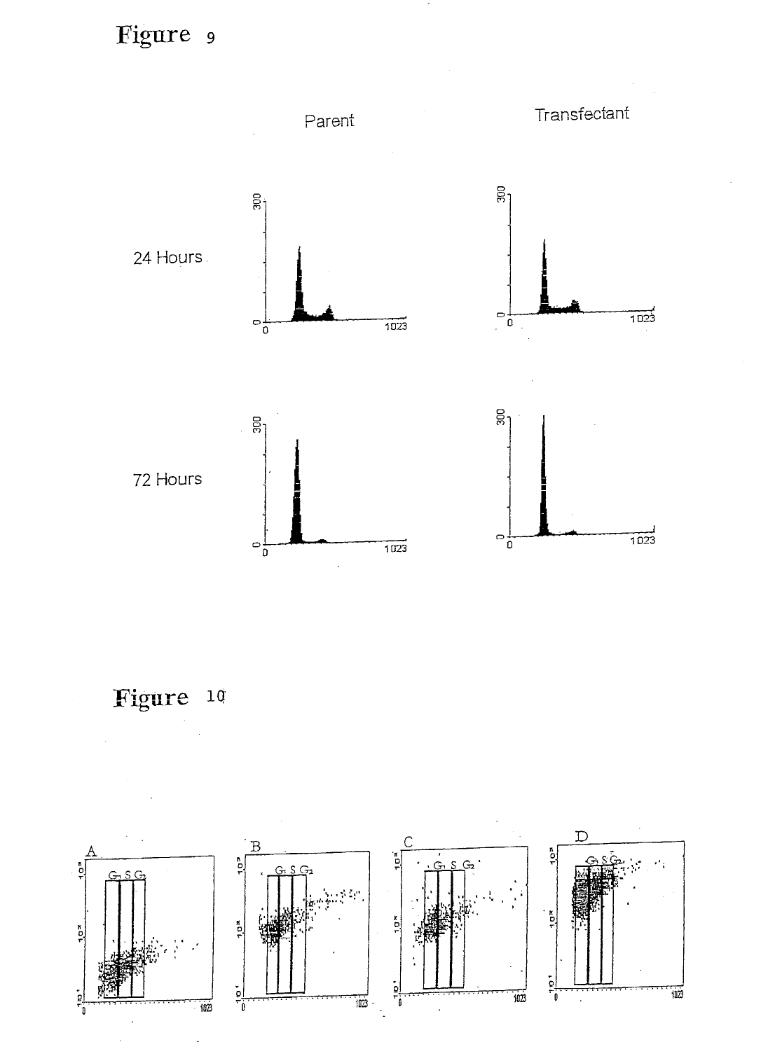

FIG. 9 shows propidium iodide histograms of uninduced and ponasterone-induced exponentially growing, asynchronously cultures of clone 2870 1D/735 at 24 h and 72 h after ponasterone induction/mock induction. It can be seen that CDK4 transfection does not alter the distribution of cells throughout the cell cycle phases G1, S and G2/M. In addition, there is no evidence of a subdiploid apoptotic cell population.

FIG. 10 shows bivariate analysis of CDK4 or CDK1 expression throughout the cell cycle in exponentially growing, asynchronous cultures of uninduced and ponasterone-induced clone 2780 1D/735. Both CDK4 and CDK1 proteins are expressed throughout the cell cycle (i.e. CDK4 expression is not limited to the G1 phase and CDK1 expression is not limited to G2 and M phase). It can be seen that in the cultures induced by ponasterone, the levels of both the CDK1 and CDK4 gene products are higher than in the corresponding uninduced cultures. Both the uninduced and induced cultures had a similar distribution of cells in each phase of the cell cycle.

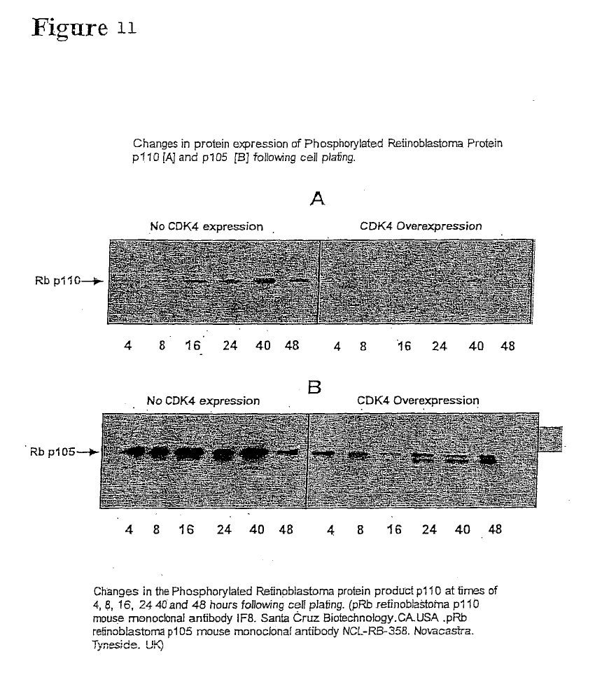

FIG. 11 shows western blots of samples of uninduced and ponasterone-induced clone 2780 1D/735 at 4, 8, 16, 24, 40 and 48 h after induction/mock induction. Panel A is probed with an antibody specific for hyperphosphorylated pRb (p110). Panel B is probed with an antibody raised against hypophosphorylated pRb (p105) that recognises both hyperphosphorylated and hypophosphorylated pRb. Panel A shows that in the uninduced clone 2780 1D/735, there is a low level of hyperphosphorylated pRb (p110). In the induced clone, hyperphosphorylated pRb (p110) is virtually undetectable. Panel B shows that in the uninduced cell line (clone 2780 1D/735 not stimulated with ponasterone), most pRb is hyperphosphorylated. At 24 h after induction, however, the induced cell line contains both hyperphosphorylated and hypophosphorylated pRb.

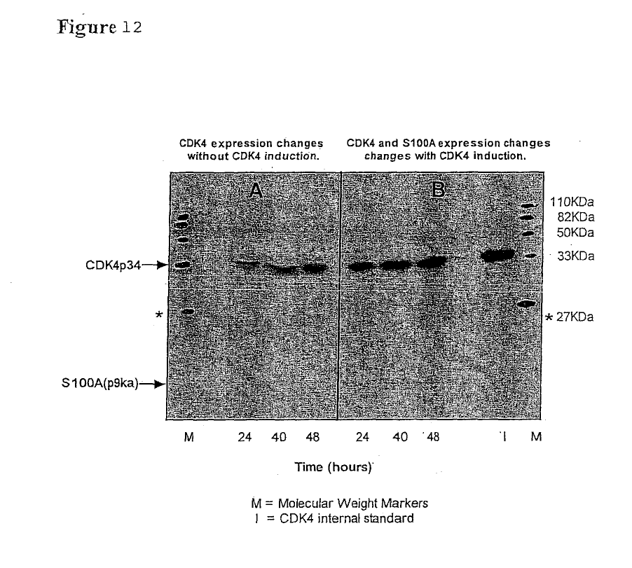

FIG. 12 shows western blots of samples of ponasterone induced and uninduced clone 2780 1D/735 at 24, 40 and 48 h after induction/mock induction. Panel A shows the blots of the uninduced samples and Panel B shows the results for the induced samples. The upper portion of each panel is probed with an antibody specific for CDK4 protein. The lower portion of each panel is probed with an antibody specific for p9Ka protein. Panel A shows that CDK4 protein (p110) is present at a low level in the uninduced samples. p9Ka protein is undetectable in the uninduced samples. In the induced samples, CDK4 protein is present at a higher level than in the uninduced samples. The level of CDK4 protein also increases with time post induction. p9Ka is detectable in the induced samples and increases with time post induction.

The present invention provides a method of screening for an agent, effective in the treatment of a cancer. The method generally comprises three steps.

The first step of the method is the selection of putative agents that are likely to disrupt a critical normal gene product. A Critical Normal Gene Product in the present context may be a gene product whose critical normal function must be retained in a cancer cell in order to enable it to continue to survive and divide successfully. Critical Normal Gene Products are defined by two characteristics. Firstly, the product of a Critical Normal Gene in the cancer sample must either be wild type (i.e. it has the same sequence as the gene product from a control sample, such as a sample of non-cancerous cells), or have no mutations that affect the critical normal function of the gene product. Where a gene product has a plurality of functions, there must be no mutations that affect a function critical to the cancer cell. The critical normal function of a critical normal gene product may be the same or different to the function of the gene product in normal cells. Secondly, the product of a Critical Normal Gene in the cancer sample is present in equal or higher amounts than in the control sample.

In the present invention, a cancer cell includes a cell taken from a primary tumour, a metastasis or other suspected site of cancer in a subject, or a cell line derived from a cancer. A preferred embodiment is where the cancer cell is derived from a primary tumour, a metastasis, blood, urine, pleural effusions, ascites, faeces, cerebrospinal fluid or another sample from a patient believed to be suffering from cancer, especially malignancy. In addition, a cancer cell includes a cell taken from a cell line derived from a cancer. A preferred embodiment is where the cancer cell sample comprises induced cells derived from a cancer cell line transfected with a critical normal gene under the control of a regulatable promoter. A control cell may be utilised in the present invention. The control cells are normal, non-cancerous cells and may be derived from the corresponding normal tissue of a patient, from any other normal tissue of a patient or from a primary cell line. In addition, the control cells may be obtained from normal tissue or blood, urine, pleural effusion, ascites, cerebrospinal fluid and faecal samples from a subject not suffering from cancer. Uninduced cells derived from a cancer cell line transfected with a critical normal gene under the control of a regulatable promoter may also be used as control cells.

In the present context, disruption of a critical normal gene product includes any disruption of a critical normal gene or the product of the gene which causes the gene and/or gene product to function incorrectly or not to function at all. Disruption of a critical normal gene includes inhibiting production of the critical normal gene product or inhibiting the activity of the critical normal gene product.

A preferred embodiment is where disruption of the critical normal gene or critical normal gene product prevents a critical normal function of that gene product. Where a critical normal gene product has a plurality of functions, disruption prevents one or more functions of the gene product critical to the cancer cell, for example successful division and continued cell survival.

Chemotherapeutic agents are preferred agents used in the screening method for effective agents that are cytotoxic to, and/or inhibiting to the growth of cancer cells. In the present invention, chemotherapeutic agents include antisense agents, ribozymes, peptides, proteins, antibodies, competitive and non-competitive inhibitors of the critical normal gene product and inhibitors of transcription or translation of the critical normal gene.

Antisense agents may be used to subtract the expression of a Critical Normal Gene. It is possible to design antisense agents to bind to a particular gene using standard techniques. One technique is to use a computer program such as Amplify to select a set of antisense oligonucleotides that bind to the RNA target and that have the following characteristics (1) length between 10 and 35 bases (2) negligible self interaction under physiological conditions (3) melting temperature less than 40° C. under physiological conditions and (4) no more than 40% of the oligonucleotide being a run of guanines or cytosines. Using a reference such as Genbank, it can be ensured that the antisense oligonucleotides have less than or equal to 85% homology with the RNA transcripts of other genes. They can be synthesised using standard procedures.

Competitive and non-competitive inhibitors of the critical normal gene product are chemotherapeutic agents that can be used in the present invention. Where the critical normal function of the gene product is known, competitive inhibitors can be produced. Competitive inhibitors are molecules that resemble a target of the gene product. For example, where the Critical Normal Gene Product is a DNA binding protein, synthetic double-stranded oligonucleotides that contain the binding site for the DNA binding domain would be putative chemotherapeutic agents. Synthetic double stranded oligonucleotides can be produced by standard methods.

The screening method further comprises steps of testing the efficacy of the putative agents in samples of control and cancer cells and identification of effective agents.

Appropriate culture conditions for the cancer and control cells are used and the cultures are treated with the putative agent. Typically, those agents that are more cytotoxic to, and/or more inhibiting to the growth of, the cancer cell sample than the control cell sample are effective agents. Where the cancer cell sample comprises a cell from a cancer cell line transfected with a critical normal gene under the control of a regulatable promoter, agents can be tested against cells derived from the uninduced and induced cell line. Those agents that are more cytotoxic to, and/or more inhibiting to the growth of the induced cell sample than the uninduced cell sample are effective agents.

In a preferred embodiment, the cancer cell sample consists of one or more cells in which the CDK1 and CDK4 gene products are co-elevated and the control cell sample consists of one or more cells in which the CDK1 and CDK4 gene products are not co-elevated. An effective agent is one that is more cytotoxic or growth inhibiting to the cancer cell sample than to the control cell sample and, in addition, disrupts the co-elevation of the CDK1 and CDK4 gene products in the cancer cell sample. However, this is not intended to be limiting.

Particularly preferred is an embodiment in which effective agents identified in the screen are further tested in an animal model system. Where the animal is afflicted with the disease, the efficacy of the treatment and the side effects may be tested. Where normal animals are used, only the side effects may be tested.

The present invention further provides an agent capable of disrupting critical normal gene product in such a manner as to be cytotoxic to, or inhibiting to the growth of, a cancer cell. Preferably, the agent is more cytotoxic to, or more inhibiting to the growth of a cancer cell than a control cell. Such an agent may be identified by the screening method outlined above. A preferred embodiment is where the CDK1 and CDK4 gene products are co-elevated in the cancer cell, but not the control cell, and wherein the agent further disrupts the co-elevation of the CDK1 and CDK4 gene products in the cancer cell.

In the present context, co-elevation of the levels of both the CDK1 and CDK4 gene products in a cancer cell is considered to occur when the level of the CDK1 gene product is statistically significantly higher than the level of the CDK1 gene product present in control cells, and the level of the CDK4 gene product is statistically significantly higher than the level of the CDK4 gene product present in control cells. Methods of measuring the levels of the CDK1 and CDK4 gene products are well known to one skilled in the art.

In a preferred embodiment, co-elevation of the levels of CDK1 and CDK4 gene product occurs when the levels of both the CDK1 and CDK4 gene products are elevated as described above, and where the ratio of the level of the CDK1 gene product to the level of the CDK4 gene product is in the range of 0.6 to 1.6.

Also preferred is an embodiment wherein said cancer is breast, prostate, colon, bladder, stomach, pancreatic or oesophagus cancer, small cell lung cancer, non-small cell lung cancer, malignant melanoma, neuroblastoma or a leukaemia.

Therefore, the present invention improves upon known methods of anticancer drug screening by providing effective drug targets. The conventional approach to anticancer drug development has identified a large number of potential drug targets, only some of which appear to be effective. In contrast, the present invention identifies a relatively few critical normal gene products which form effective drug targets. The drug targets are likely to be effective because the critical normal gene products, by definition, are wild type (or have no mutations that affect a critical normal function of the gene products in a cancer cell) and present at equal or higher amounts than in normal cells, and therefore, the target is known to be present in the tumour. In addition, the screen of the present invention is not limited to targeting genes and gene products involved in early carcinogenesis. The screen targets gene products that play a role in the full malignant phenotype.

The present invention also solves the problems of tumour heterogeneity and genetic instability that complicated previous cancer therapies. Critical normal gene products whose function is essential for continued cancer cell survival and proliferation are likely to be homogeneous and stable throughout the tumour cell population. Loss of function of such critical normal gene products would remove the critical function they provided and result in cancer cell death. These gene products would thus be expected to be present and functional in every tumour cell and therefore provide a consistent anticancer drug target, unaffected by tumour heterogeneity and genetic instability. Drug resistance would thus not be a problem with new agents developed against critical normal gene product targets.

The invention also provides a pharmaceutical composition comprising an agent as described above and medicinal uses of the agent and pharmaceutical composition.

Also provided is a method of treating a patient having cancer. Such a treatment is tailored to the particular cancer and is more effective than known treatments. The method comprises the following steps:

a) identifying a critical normal gene present in said cancer;

b) treating the patient with an agent or pharmaceutical composition capable of disrupting said critical normal gene product in such a manner as to be cytotoxic to, or inhibiting to the growth of, a cancer cell.

Preferably, the agent or pharmaceutical composition is more cytotoxic to, and/or more inhibiting to the growth of a cancer cell sample than a control cell sample.

The first method step is identification of the Critical Normal Genes present in the cancer. This step involves comparing putative Critical Normal Gene products in a sample of cancer cells with control cells or an extract therefrom. As mentioned above, Critical Normal Gene Products are wild type in cancer cells (i.e. the gene product in the cancer sample has the same sequence as the gene product from the control sample), or has no mutations that affect the function critical to continued cancer cell survival and proliferation. Where a gene product has a plurality of functions, the gene product contains no mutations that affect the function critical to the cancer cell, for example successful cell division and continued cell survival. The critical normal function of a gene product may be totally distinct from the function of the gene product in normal cells. In addition, a Critical Normal Gene Product is present in equal or higher amounts in the cancer sample than in the control sample.

Gene products may be either RNA or protein. In the case of a gene that gives rise to a protein product, mRNA is produced as an intermediate. In such a case, either the mRNA or the protein can be tested to see whether the criteria for a Critical Normal Gene are met.

In the case where the gene product is an RNA, a three step test to identify a Critical Normal Gene is preferably employed. The first step is to measure the level of the gene product in both cancer and control samples. Determination of RNA levels can be effected in a number of ways (e.g. Northern blotting; Slamon D J, Dekernion J B, Verma I M, Cline M J, Expression of cellular oncogenes in human malignancies, Science 224, 256-262) and would be routine to a person skilled in the art. One can readily convert poly-A bearing mRNA to cDNA using reverse transcription. It is also possible to reverse transcribe RNA without a poly-A tail by first ligating a poly-A tail to the 3′ end of the RNA molecule. Reverse transcription PCR methods allow the quantity of single RNAs to be determined, but with a relatively low level of accuracy. Arrays of oligonucleotides are a relatively novel approach to nucleic acid analysis, and can be used to accurately measure the quantity of an RNA (Pease et al. (1994) Proc Natl Acad. Sci USA 91:5022-5026; Maskos and Southern (1993) Nucleic Acids Research 21: 2269-2270; Southern et al. (1994) Nucleic Acids Research 22: 1368-1373). The levels of the gene products in the two samples can then be compared. If the RNA is a Critical Normal Gene product, the levels of RNA in the cancer sample must be greater than or equal to those in the control sample. The second step is to identify the sequence of the gene in both cancer and control samples. This can be done by sequencing cDNA produced from both samples by means of reverse transcription (see above). Also, arrays that measure both the expression levels of RNAs and detect mutations in those RNAs are being developed. Such arrays offer an attractive means to identify critical normal genes. The sequences should be compared. For the RNA to be the product of a Critical Normal Gene, the RNA from the cancer sample must either be wild type (i.e. have the same sequence as the RNA from the control sample) or contain no mutations that affect the critical functioning of the gene product.

In the case where the putative Critical Normal Gene Product is a protein, the first step to identify a Critical Normal Gene is to measure the protein levels in each sample. Methods of measuring protein levels would be well known to one skilled in the art. Measuring protein levels can be achieved by ELISA (Voller A, Bidwell D E, Bartlett A, ‘The Enzyme Linked Immunoabsorbent Assay (ELISA). A guide with abstracts of microplate applications’, Nuffield Laboratories of Comparative Medicine, the Zoological Society of London, Regent's Park London NW1. 1979. ISBN 0.906036.01.1. Sponsored by and available from Dynatech Europe, Borough House Rue du Pre, Guernsey, G B), Western Blotting (Stryer L, Exploring proteins Chapter 3 in Biochemistry 4^{th} Edition (1995) Ed. Lubert Stryer, W H Freeman and Company New York), FACS analysis/flow cytometry (Watson J, Stewart J, Cox H, Evan G, Sikora K, ‘Flow cytometric quantisation of the c-myc oncoprotein Mol Cell Probes’, 1, 1151-158) or immunocytochemistry (Stratton M R, Gusterson B, ‘New techniques in pathology and their application in diagnosis and studies of tumour biology’, Chapter 3.2 pp 350-362 in The Oxford Textbook of Oncology Volume 1 (1995) Eds. Michael Peckham, Herbert Pinedo and Umberto Veronesi, Oxford University Press, Oxford, N.Y., Tokyo). Proteomic chips and biosensor techniques may also be used in the present invention to measure protein levels. These techniques are both fast and sensitive.

The levels of the protein in the two samples can then be compared. If the protein is a Critical Normal Gene Product, the level of protein in the cancer sample must be greater than or equal to that in the control sample. The second step is to identify the sequence of the gene in both cancer and control samples. This can be done by either sequencing the gene as above, or by the use of antibodies specific to the wild type protein. For the protein to be the product of a Critical Normal Gene, the gene from the cancer sample must either be wild type (i.e. have the same sequence as the gene from the control sample) or contain no mutations that affect the critical functioning of the protein in a cancer cell.

Examples of Critical Normal Genes identified using these methods include cyclin dependent kinase inhibitor p27^{KIP1}, retinoblastoma, CDK1, CDK4 and telomerase.

For providing treatment tailored to a cancer patient, it is necessary to identify a critical normal gene that is present in the cancer. It is then possible to administer an agent capable of disrupting this critical normal gene or a pharmaceutical composition comprising this agent to the patient.

In a preferred embodiment, two or more critical normal genes are identified in a cancer and the patient is treated with agents capable of disrupting the two or more critical normal genes identified.

Also preferred is an embodiment wherein said cancer is breast, prostate, colon, bladder, stomach, pancreatic or oesophagus cancer, small cell lung cancer, non-small cell lung cancer, malignant melanoma, neuroblastoma or a leukaemia. The cancer may be metastatic or non-metastatic.

The invention provides a method of selecting a suitable treatment for a cancer patient from known treatments, which method comprises identification of a critical normal gene present in said cancer as described above, followed by selecting a known treatment that disrupts the function of said critical normal gene product which is critical to cancer cell survival and proliferation.

The invention also provides a method for treating a cancer in which the CDK1 and CDK4 gene products are co-elevated comprising treating the patient with an agent that is capable of disrupting a critical normal gene product in such a manner as to be cytotoxic to, or inhibiting to the growth of a cancer cell and which further disrupts the co-elevation of the CDK1 and CDK4 gene products. Preferably, the agent is more cytotoxic to, or more inhibiting to the growth of a cancer cell than a control cell.

Thus the present invention advantageously provides for selecting a treatment for a patient having cancer that is tailored to the genetic makeup of the cancer. This improves the efficacy of the treatment. In addition, it reduces the cost and delay resulting from selecting treatments that are not suitable for the cancer.

The present invention further provides a kit for selecting and providing a suitable treatment for a patient having a cancer, which kit comprises:

a) a means for identifying a critical normal gene present in said cancer; and

b) an agent capable of disrupting a critical normal gene product in such a manner as to be cytotoxic to, or inhibiting to the growth of said cancer.

Preferably, the agent is more cytotoxic to, or more inhibiting to the growth of said cancer cell than a control cell.

Preferably, the means for identifying a critical normal gene is an antibody recognising the product of said critical normal gene.

Another embodiment of the invention provides a method of screening for an agent effective in preventing a cancer from undergoing metastasis, which method comprises:

a) selecting a putative agent that is likely to disrupt a critical normal gene product present in said cancer;

b) determining the ability of a sample of metastatic cancer cells to undergo metastasis in the presence of said agent; and

c) identifying an effective agent as an agent which, when present, reduces the ability of said sample of metastatic cancer cells to metastasise;

wherein said putative agent is a peptide or protein.

Preferably, the ability of the sample of metastatic cancer cells to metastasise is determined in the absence of said agent. The ability of metastatic cells to metastasise is reduced in the presence of an effective agent, but not in the absence of an effective agent.

Chemotherapeutic agents are preferred agents used in the screening method for effective agents that inhibit metastasis. In the present invention, chemotherapeutic agents include peptides and proteins. The peptides and proteins may be antibodies, competitive and non-competitive inhibitors of the critical normal gene product and inhibitors of transcription or translation of the critical normal gene.

As mentioned before, in this invention Critical Normal Gene Products are defined by two characteristics. Firstly, the product of a critical normal gene in the cancer sample must either be wild type or have no mutations that affect the critical normal function of the gene product. Where the gene product has a plurality of functions, there must be no mutations that affect a function critical to a cancer cell, for example metastasis. Secondly, the product of the critical normal gene must be present in equal or normal amounts than in the control sample. Critical Normal Gene Products may be required for cancer cells to undergo metastasis. p9Ka is a critical normal gene product that is required for metastasis.

RAMA37 cells transfected with CDK4 undergo metastasis (Table 1). As the CDK1/CDK4 gene products are coelevated in the majority of cancer cells and as mutations in CDK4 are very rare (Example 4), CDK4 may act as a critical normal gene for metastasis.

Osteopontin is another normal protein that has been reported to be upregulated in cancer cells and to influence prognosis in prostate cancer (Hotte et al. (2002) Cancer 95: 506-512), breast cancer (Rudland et al. (2002) cancer Res. 62: 3417-3427), and a number of other tumours. Osteopontin acts as a critical normal gene in these cancer cells to promote metastasis.

A sample of metastatic cancer cells may comprise one or more cancer cells derived from a metastasis present in subject suffering from cancer. A sample of metastatic cancer cells may comprise one or more cancer cells derived from a primary cancer or a cancer cell line that metastasises when introduced into an animal.

The first step of the screening method is selecting an agent which is likely to disrupt a critical normal gene product. Chemotherapeutic agents and antisense agents are preferred agents in this embodiment. These may be designed to disrupt critical normal genes as described above.

The second step of the screening method involves determining the ability of a cancer cell sample to undergo metastasis in the presence of the agent. The method used to determine the ability of a cancer cell sample to undergo metastasis in the presence of an agent is not particularly limited and any suitable method may be used. In a preferred embodiment, the sample of cancer cells is injected into an animal model such as a rat. The agent is then administered to the animal model. The dosage of the agent may be determined by one skilled in the art. After a suitable period of time, the length of which may be determined by one skilled in the art, the animal is sacrificed and any metastases are identified. In a preferred embodiment, the animal model used is the nude mouse model described by Kojonniksen and colleagues (Kojonniksen et al. (1994) Cancer Res. 54: 1715-1719) or the nude rat model described by Weterman and co-workers (Weterman et al. Cancer Res. 52: 1291-1296).

The present invention also provides an agent capable of disrupting a critical normal gene product in such a manner as to reduce the ability of metastatic cancer cells to metastasise. Such an agent may be identified by the screening method for agents effective in inhibiting metastasis. The invention also provides a pharmaceutical composition comprising an agent as described above and medical uses of the agent and pharmaceutical composition.

Also provided is a method a preventing metastasis of a cancer in a patient, which method comprises:

a) identifying a critical normal gene product present in said cancer; and

b) treating the patient with an agent or pharmaceutical composition capable of disrupting said critical normal gene product.

Critical normal genes may be identified as outlined above. Once a critical normal gene has been identified, it patients may be treated with an agent that disrupts said critical normal gene. This agent may be a known agent or an agent identified in the screen for effective agents.

The invention also provides a kit for selecting an agent for preventing metastasis of a cancer comprising:

a) a means for identifying a critical normal gene present in said cancer; and

b) an agent capable of disrupting a critical normal gene.

Another aspect of the invention is concerns a method for analysing gene expression in dying cells. In particular, the invention provides an additional method for identifying a critical normal gene which method involves comparing the levels of a gene product in dying and proliferating cancer cells. The method comprises:

a) detection of a gene product in L23COR cells that are quiescent or proliferating;

b) detection of said gene product in dying L23COR cells; and

c) identifying a critical normal gene as a gene product which is present at higher levels in quiescent or proliferating L23COR cells than in dying L23COR cells.

In the present invention, L23COR cells that are quiescent or proliferating encompasses L23COR cells within the cell cycle and L23COR cells in Go i.e. cells which are not dying. Quiescent or proliferating cancer cells are adherent to the surface of the tissue culture plate when grown in tissue culture. Dying L23COR cells do not adhere to the surface of the tissue culture plate when grown in tissue culture. One skilled in the art can separate adherent and non-adherent cells and can therefore separate quiescent or proliferating L23COR cells from dying L23COR cells. Gene expression in substantially pure populations of quiescent or proliferating L23COR cells, or dying L23COR cells can then be analysed by standard methods.

Typically, detection of a gene product will take place by FACS analysis, immunocytochemistry, Western Blotting or Northern Blotting, however, the method of detection is not particularly limited and any suitable method may be used.

The applicant arrived at the present invention by studying the genetic makeup of clinical cancers. The applicant realised that the severe damage in the cell division, differentiation, senescence and death pathways in the cells of a clinical tumour that progressively occurs as the cancer evolves could potentially disrupt the normal function of these pathways so much that the cells become unable to undergo further cell division. The applicant identified a class of genes which appear to be essential for the continued survival and proliferation of a cancer cell which are now termed “Critical Normal Genes”. These genes remain wild type or contain no mutations that would affect a function of their product in cancer cells that is critical to the cancer cell, for example continued cancer cell survival and division. The products of such Critical Normal Genes assume a much more important role in the chaotic molecular environment of a typical clinical cancer cell than in a normal diploid cell. This is because in normal cells, the mechanisms controlling cell division, differentiation, senescence and death involve multiple, parallel positive and negative signalling pathways from the cell surface that interact with the molecular machinery controlling the cell cycle (Jones and Walker (1999) Mol Pathol. 52: 208-213; Hill (1999) Int. J Biochem Cell Biol. 31:1249-1254; Miller et al. (1999) Oncogene 18:7860-7872; McCormick (1999) Trends Cell Biol. 9: 53-56; Lloyd et al. (1997) Genes and Development 11: 663-677). There is cross-talk between the components of these pathways which therefore function as a network (Mihich and Harlow (2000) Cancer Research 60: 7177-7183; Bhalla and Iyengar (1999) Science 283: 381-390; Kohn (1998) Oncogene 16: 1065-1075). So, in normal diploid cells, any individual critical normal gene would have a small role in the multiple interacting pathways controlling cell division and death. The progressive genetic damage that accrues during carcinogenesis results in cells in which the molecular mechanisms controlling cell growth and survival are not necessarily the same as those of normal diploid cells. This is known as bizarre cell circuitry (Weinstein (2000) Carcinogenesis 21:857-864). As fewer genes are involved, those gene products, whose continued normal functions are required for cancer cells to be able to divide, assume a greater importance as there is less redundancy.

The applicant realised that Critical Normal Genes and Critical Normal gene products responsible for cancer cell survival and proliferation are potential anticancer drug targets because they are homogeneous and stable throughout the tumour cell population. If they were damaged, this would remove the critical function they provided, resulting in cancer cell death or a reduction in cancer cell proliferation. These gene products would thus be expected to be present and functional in every tumour cell and therefore provide a consistent anticancer drug target unaffected by tumour heterogeneity and genetic instability.

Given the considerable phenotypic variations in human clinical cancers, although Critical Normal Gene Products may exist, they may not always provide ubiquitous targets. The applicant realised that it would be necessary to identify what Critical Normal Genes are present in a cancer in order to select a suitable treatment.

Similarly, the applicant realised that, due to the large number of mutations occurring, other processes carried out by cancer cells such as metastasis may also be affected by the large number of mutations present. The applicant identified gene products that must remain intact for metastasis to occur. Such critical normal genes for metastasis can be targeted to inhibit metastasis. As the majority of deaths from cancers of solid tissues are attributable to the process of metastasis, prevention of metastasis may greatly improve a cancer patient's prognosis.

Gene products often have numerous functions. Where this is the case, a critical normal gene product or a critical normal gene for metastasis may bear mutations in a region that is not responsible for mediating the critical normal function. The critical normal function of a gene product in a cancer cell may be distinct from the function of the gene product in normal cells.

The CDK4 gene product has numerous functions; these include binding to cyclin D, binding to and phosphorylating pRb, binding to cyclin dependent kinase inhibitors such as p21, p27, p16 and other members of these families of CDKIs, interacting with a cyclin activating kinase and interacting with enzymes responsible for phosphorylating and dephosphorylating tyrosine 17. In cancer cells, induction of CDK4 does not act to induce cells to progress from G1 to S phase and does not lead to phosphorylation of Rb. In fact, increasing levels of CDK4 protein increases the amount of hypophosphosrylated pRb. Therefore, CDK4 protein does not appear to perform its normal functions in cancer cells. This implies that some other function of CDK4 protein is critical to the survival of cancer cells.

The applicant has identified a novel function of the CDK4 gene product. The CDK4 gene product acts to elevate the level of CDK1 in cancer cells. The pathway by which the CDK4 gene product elevates the CDK1 gene product is not yet fully known but, without being bound by theory, appears to be independent of its classical pathway of phosphorylating pRb. It is therefore a good candidate for mediating the critical normal function of the gene product. Without being bound by theory, the applicant considers that in the early stages of carcinogenesis, the CDK4 gene product has the same role as in normal cells. However, in cancer cells where there is severe disruption of normal function, the CDK4 gene product takes on a new role of promoting survival of otherwise very damaged cells by elevating levels of the CDK1 gene product. The CDK2, p27, p21, and Rb gene products are also co-elevated in human colorectal cancer. The applicant considers that the CDK4 gene product may also be responsible for upregulation of these gene products in cancer cells. The proposed mechanism of CDK4 in cancer cells is shown in FIG. 2. Many of the gene products upregulated by CDK4 are critical normal gene products. The applicant considers that this function of the CDK4 gene product may be critical to cancer cells in that it increases expression of other critical normal genes. These critical normal genes include those responsible for cancer cell survival and proliferation. CDK1, p27 and Rb are critical normal genes that are required for cancer cell survival and proliferation. The applicant considers that agents that disrupt this critical normal function of CDK4 may be effective anti-cancer agents.

Overexpression of CDK4 protein may also lead to metastasis. Elevation of CDK4 induces metastatic capability in RAMA37 cells (Table 1). In addition, overexpression of CDK4 protein leads to the elevation of the p9Ka protein. p9Ka protein has been shown to induce metastatic capability in non-metastatic mouse and rat mammary tumour cells (Davies et al. Oncogene (1993) 8: 999-1008; Grigorian et al. Int. J. Cancer (1996) 67: 831-841) and elevation of this protein may lead to metastasis.

Overexpression of CDK4 protein also leads to the elevation of critical normal gene products required for angiogenesis. Critical normal gene products required for metastasis include VEGF protein, FGF protein and TGF protein.

Analysis of the amino acid sequence of CDK4 indicates that a region near to the C-terminus (between 172 and 285) has no known function. The applicant postulates that this may be the region of the CDK4 gene product that mediates the novel critical normal function.

The experiments that lead the applicant to deduce that there is a novel critical normal function of CDK4 are described below. Details of the protocols used in the experiments are not intended to be limiting.

Experiment 1—Generation of Human Expression Vectors Containing the CDK4 Open Reading Frame and a Human Ovarian Cancer Cell Line Containing the Expression Vector

The human ovarian carcinoma cell line 2780 has amplified the CDK4 gene. It was therefore postulated that CDK4 mRNA would be abundant in this cell line. RNA was extracted from the cell line using the Tri Reagent (Sigma T9424) according to the manufacturer's instructions. The extracted RNA was then quantified spectrophotometrically. 1 μg of the RNA was reverse transcribed with SuperScript RT RNase H-Reverse Transcriptase (Gibco BRL, 18053-17) using oligo (dT) as a primer. The resultant cDNA was used as a template for amplification of CDK4 DNA by reverse transcription PCR.

Primers were designed to flank CDK4 cDNA. These primers were designed to incorporate the restriction enzyme sites EcoRI (Roche 1175 084) and NotI (Roche 1014 714) to allow the subsequent directional cloning of the amplified gene product into the multiple cloning site of the pcDNA3 vector (Invitrogen V790-20). The primers were synthesised by MWg Biotech.

5′ CCC GAA TTC AGA ATG GTC ACC TCT CGA TAT GA 3′

3′ CCC GCG GCC GCT GCT CAC TCT GGA TTA CCT T 5′

5 μl cDNA was amplified in 50 μl PCR reactions containing 20 pmoles of each primer using Taq DNA polymerase (Gibco BRL 18038-026). The reactions were placed in a pre-heated PCR block at 95° C. for 2 minutes before undergoing 30 cycles of denaturation (30 s at 95° C.), annealing (30 s at 65° C.) and extension (1 min at 72° C.). The length of the PCR products was checked on a 1% (w/v) agarose gel before being purified using Qiaquick PCR purification kit (Qiagen 28104). The purified DNA was used directly in sequencing reactions to ensure the whole wild-type CDK4 open reading frame was amplified.

Sequencing primers (10 pmoles), identical to those employed in PCR reactions were radioactively labelled at their 5′ ends with γ³²P-ATP (45 μCi). Sequencing was carried out using the f-mol DNA sequencing system (Promega-Q4100). It was found that the entire wild type CDK4 open reading frame had been amplified.

The CDK4 DNA and the plasmid vector pIND were digested sequentially with EcoRI and NotI according to the manufacturer's instructions. Bacteriophage T4 DNA ligase (New England Biolabs 202L) was used to ligate the digested CDK4 DNA to digested pIND. The ligated vector was used to transform E. coli. Transformants were selected by plating onto agar containing ampicillin. Colonies containing recombinant plasmids were identified by colony PCR and restriction analysis of minipreps of plasmid DNA, and then purified using the Concert Midi Prep Kit (GibcoBRL 11451-010).

The mammalian expression vector pVgRxR (Invitrogen) was transfected into human ovarian cancer cell line 2780 using lipofection (lipofectamine plus Gibco cat no. 10964-013) or fuGENE 6 (Roche cat no 1-814 443) according to the manufacturer's instructions. Clones of cells surviving geneticin (G418 Gibco-BRI 10131-019) selection were then transfected with the pIND CDK4 construct, again using lipofection. Clones of cells surviving selection with zeocin (Invitrogen 45-0380) were then screened by PCR to check that they contained the CDK4 gene using the specific primers for the ecdysone/glucocorticoid response element. One clone generated in this manner was named 2780 1D/735. This clone is used in many of the following experiments.

Experiment 2—Determination of the Expression Levels of the CDK1 and CDK4 Gene Products Following Induction of 2780 1D/275

The level of CDK4 protein and CDK1 protein present in high, medium and low CDK4-expressing clones generated as described in Experiment 1 was determined at 24, 48 and 72 h following induction of CDK4 expression with ponasterone. At various time points after induction, the induced cells were harvested using Trypsin/Versene (Sigma). The harvested cells were centrifuged at 250G for 5 min. The supernatant was decanted and the pellet was resuspended in PBS and the centrifugation was repeated. The cells were then lysed at 3×10⁷ cells per ml using a denaturing lysis buffer. The denaturing buffer was prepared by dissolving one tablet of complete Mini™ (Roche), a protease inhibitor cocktail, in 10 ml of a solution of 1% w/v SDS, 0.8% v/v Glycerol, 0.05M Tris (pH 6.8). The resultant lysate was then sonicated on ice for 10 seconds, and this solution was then centrifuged at 17,500G for 30 minutes at 4° C. The supernatant was then aliquoted, and stored at −80° C.

The protein content of each lysate was determined using a Micro-BSA Protein Determination Kit (Pierce), and the lysates were run on a discontinuous gel of 4%T, 2.6%C stacking gel, with a 10%T, 2.6%C resolving gel using the Lammelli system.

The resolved proteins were then transferred to a nitrocellulose membrane (Protran, Schleicher & Schuell) by electroblotting. Following electrophoretic transfer the membrane was ‘blocked’ for 1 hour with 10%w/v Marvel in TBS at room temperature. The membrane was then probed using a two-stage antibody detection method. The first stage used antibodies generated to human CDK1^{cdc2} (cdc2 p37 (17), Santa Cruz, cat. no. Sc-54) and human CDK4 (CDK4 (c-22), Santa Cruz, cat. no. Sc-260). The second stage involved the subsequent probing of the membrane using Horse Radish Peroxidase conjugated Sheep anti-mouse IgG (Serotec, cat. no. AAC10P) for CDK1, and Horse Radish Peroxidase conjugated Sheep anti-rabbit IgG (Serotec, cat. no. STAR54) for CDK4. The protein bands were then detected using Luminol based Chemoluminescent reagent ECL™ Western blotting detection reagent Kit (from Amersham pharmacia biotech, cat. no. RPN2106), and Hyperfilm™ MP (from Amersham pharmacia biotech, cat. no. RPN1675K). The protein band signals obtained following probing of the membranes as described above were then analysed using the Phoretix software system (Phoretix 1D Gel Analysis, from Non Linear Dynamics Ltd).

The results are presented in FIG. 6. The figure clearly shows that the level of the CDK1 gene product increases as the level of CDK4 rises within the cells of each clone. The figure also shows that the levels of the CDK1 and CDK4 gene products are present in an approximately 1:1 ratio. FIGS. 4 and 5 show that the levels of these proteins are not related in normal fibroblast and keratinocyte cells. Without being bound by theory, the applicant hypothesises that the mechanism by which the CDK4 gene product elevates the level of the CDK1 gene product includes a feedback mechanism that ensures that the levels of the two proteins are approximately equal.

Experiment 3—ELISA Assay Showing the CDK1 and CDK4 Gene Products are Co-Elevated in 2780 1D/275 Following Induction

Cells of clone 2780 1D/275 were plated at a density of 20000 cells/well into a 96 well flat bottomed TC plate (Costar) in 100 μl of medium (Hams F12 medium+5 mM Glutamine) containing 10% FBS. The plate was incubated overnight at 37 C in 5% CO2. 100 μl medium plus 10% FBS plus 5 μM pontasterone was then added to the test wells. 100 ml medium plus 10% FBS was added to control wells. The plate was then incubated as above for 48 hours. The medium was then removed and each well was washed three times with 100 μl PBS. The samples were then fixed for 30 minutes by addition of 2% paraformaldehyde in PBS. The wells were then washed three times in ELISA wash buffer (40 g NaCl, 1 g KCl, 14.5 g Na₂HPO₄.12H2O, 1 g KH₂PO4 in 5L distilled water). Each wash lasted 5 minutes. The wells were blocked with 1% BSA in ELISA wash buffer for 20 min. The wells were then washed once with wash buffer (Standard ELISA wash Buffer containing 0.1% Tween 20).

To measure the level of CDK4 protein produced by the ponasterone induced cells, a first antibody solution consisting of 100 μl of a 1 in 1000 dilution of anti-CDK4 (Santa Cruz Rabbit polyclonal to CDK4) in PBS was added to the test wells. No first antibody was added to the control wells. The plate was then incubated for 1 hour at 37 C. The cells were then washed three times with wash buffer. Each wash lasted 5 min. To measure the degree of binding of the first antibody, 100 μl of a second antibody solution consisting of a 1 in 1000 dilution of a Goat anti-Rabbit Horse Radish Peroxidase Conjugated antibody was added to each well (both test and control wells) and the plate was incubated for 1 h at 37° C. The wells were then washed three times with wash buffer as described above. The cells were developed with DAB (Dako DAB Tablets) DAB for 30 min. The reaction was stopped with 4% H₂SO4. Each well was read at 450 nm on Biorad plate reader. The experiments were repeated using anti-CDK1 as the primary antibody.

FIG. 7 shows that the CDK1 and CDK4 gene products are co-elevated in 2780 1D/275 48 h after ponasterone induction of transfected exogenous CDK4 DNA.

Experiment 4—CDK1/CDK4 Ratio in Proliferating and Dying Human Cancer Cells

L23COR (a human non-small cell lung cancer cell line) was grown as a monolayer attached to the bottom of a tissue culture flask. The cells shed from the confluent monolayer were collected, transferred to a flask and their fate was followed for several days. The detached cells underwent spontaneous cell death. Detached L23COR cells provide a model for dying human cancer cells. This model was used to analyse the molecular changes that occur when human cancer cells die.

L23COR cells were cultured. The culture medium (containing the detached cells) was removed and centrifuged at 250G for 5 min. The adherent cells were washed with medium and then harvested using Trypsin/Versene (Sigma). The harvested cells were centrifuged at 250G for 5 min. Both the adherent cell pellet and the detached cell pellet was resuspended in PBS and the centrifugation was repeated. The cells were then lysed at 3×10⁷ cells per ml using a denaturing lysis buffer. The denaturing buffer was prepared by dissolving one tablet of complete Mini™ (Roche), a protease inhibitor cocktail, in 10 ml of a solution of 1% w/v SDS, 0.8% v/v Glycerol, 0.05M Tris (pH 6.8). The resultant lysate was then sonicated on ice for 10 seconds, and this solution was then centrifuged at 17,500G for 30 minutes at 4° C. The supernatant was then aliquoted, and stored at −80° C.

The protein content of each lysate was determined using a Micro-BSA Protein Determination Kit (Pierce), and the lysates were run on a discontinuous gel of 4%T, 2.6%C stacking gel, with a 10%T, 2.6%C resolving gel using the Lammelli system.

The resolved proteins were then transferred to a nitrocellulose membrane (Protran, Schleicher & Schuell) by electroblotting. Following electrophoretic transfer the membrane was ‘blocked’ for 1 hour with 10%w/v Marvel in TBS at room temperature. The membrane was then probed using a two-stage antibody detection method. The first stage used antibodies generated to human CDK1^{cdc2} (cdc2 p37 (17), Santa Cruz, cat. no. Sc-54) and human CDK4 (CDK4 (c-22), Santa Cruz, cat. no. Sc-260). The second stage involved the subsequent probing of the membrane using Horse Radish Peroxidase conjugated Sheep anti-mouse IgG (Serotec, cat. no. AAC10P) for CDK1, and Horse Radish Peroxidase conjugated Sheep anti-rabbit IgG (Serotec, cat. no. STAR54) for CDK4. The protein bands were then detected using Luminol based Chemoluminescent reagent ECL™ Western blotting detection reagent Kit (from Amersham pharmacia biotech, cat. no. RPN2106), and Hyperfilm™ MP (from Amersham pharmacia biotech, cat. no. RPN1675K). The protein band signals obtained following probing of the membranes as described above were then analysed using the Phoretix software system (Phoretix 1D Gel Analysis, from Non Linear Dynamics Ltd).

FIG. 8 shows that the level of the CDK1 gene product in the detached (dying) cells is lower than that in the adherent (proliferating) cells. In contrast, the level of the CDK4 gene product in the detached cells is approximately twice as high as the level of the CDK4 gene product in adherent cells. Without being bound by theory, the applicant hypothesises that in cancer cells, the CDK4 gene product upregulates the CDK1 gene product. The CDK1 gene product (directly or indirectly) forms part of a negative feedback loop that regulates the levels of the CDK4 gene product. Therefore, in normal cancer cells, the ratio between the levels of the CDK1 and CDK4 gene products is in the range of 0.6 to 1.6. FIG. 8 shows that proliferating L23COR cells exhibit a CDK1/CDK4 ratio of approximately 1.0. When cancer cells die, the process by which CDK4 elevation results in CDK1 elevation is disrupted. As CDK1 levels reduce, the levels of CDK4 increase unchecked distorting the ratio of the CDK1 and CDK4 gene products. FIG. 8 shows that the detached cells exhibit a CDK1/CDK4 ratio of about 0.3. The detached cells may die because the level of the CDK1 gene product is insufficient to phosphorylate survivin at mitosis leading to apoptosis (see Example 4).

This data supports the model that co-elevated CDK1/CDK4 performs a critical normal function in cancer cells, which, when lost leads to death of the cancer cells.

Experiment 5—Cell Cycle Distribution of Induced and Uninduced 2780 1D/275

The cell cycle distribution of uninduced and ponasterone-induced 2780 1D 275 was analysed using flow cytometry.

Asynchronously growing monolayers of uninduced and induced 2780 1D/275 at 24 h and 72 h after induction/mock induction were harvested using Trypsin/Versene solution (Sigma). The cell suspension was then centrifuged in Falcon tubes (Becton Dickinson) at 250G, for 5 minutes. The supernatant was then removed by pipette. The cell pellet was then resuspended in PBS, and the sample re-centrifuged. The supernatant was then removed by pipette, and the pellet resuspended by passing through a 200 μl Finnpipette tip repeatedly. The cells were then fixed in 70%v/v Ethanol in water, and stored at 4° C. overnight.

The fixed samples were centrifuged at 250G for 5 minutes, and the supernatant was removed by pipetting. The cell pellet was then resuspended in PBS, and then centrifuged for 5 minutes at 250G. The supernatant was then removed by pipetting, and the cells stained in a solution containing 20 ug.ml⁻¹ RNase A, and 10 ug.ml⁻¹ Propidium iodide. The samples were then incubated for 30 minutes at 37° C.

Stained samples were then analysed using a Becton Dickinson FACScan, using a 488 nm Argon laser, with the Lysis II data acquisition software running of Consort 32.

FIG. 9 shows that the uninduced and induced cultures have similar DNA content profiles at 24 and 72 hours following induction. It can be seen that at 24 h after induction, both the uninduced and induced culture have equal numbers of (˜8 times as many) cells in G1 as G2. After 72 hours, this ratio has increased to 34 in both induced and uninduced 2780 1D cells. Thus, CDK4 expression does not appear to alter the cell cycle distribution.

Experiment 6—Bivariate Analysis of CDK4 and CDK1 Expression Throughout the Cell Cycle

Asynchronously cultured monolayers of uninduced and ponasterone induced 2780 1D/275 were harvested using Trypsin/Versene (Sigma), and the cell suspension was the centrifuged at 250G for 5 minutes in a Falcon Tube (Becton Dickinson). The supernatant was then removed by pipetting, and the cell pellet resuspended in PBS. The sample was then centrifuged for a further 5 minutes at 250G. The supernatant was again removed by pipetting and the cell pellet was resuspended by repeated passage through a 200 μl Finnpipette tip. The cells were then fixed in Methanol overnight at −20° C.

The Methanol fixed samples were centrifuged, the supernatant was discarded, and the pellet resuspended in PBS. The cell suspensions were then centrifuged at 250G for 5 minutes, and the supernatant removed by pipetting.

The cell pellet was then resuspended by gentle agitation, and the cells were then stained using 2 μg of rabbit anti human CDK4 FITC conjugated antibody (Cdk4 (C-22) FITC, cat no. sc-260 FITC, Santa Cruz Biotechnology) per 1×10⁶ cells, or 2 μg of mouse anti human CDK1 FITC conjugated antibody (Cdc2 p34 (17) FITC, cat no. sc-54 FITC, Santa Cruz Biotechnology) per 1×10⁶ cells. The cells were then incubated at room temperature for 1 hour in the dark.

The samples were then counter-stained with 5 μg of Propidium Iodide, and incubated for 30 minutes with 20 μg of RNase A. Stained samples were then analysed using a Becton Dickinson FACScan, using a 488 nm Argon laser, with the Lysis II data acquisition software running off a Consort 32. Data was collected on the FL3 channel (red fluorescence) to determine the DNA content of the cell population, and FL1 channel to determine the level and distribution of the CDK1 and CDK4 proteins.

The List Mode Data obtained from the FACScan was translated into DOS using HP-LIF to DOS conversion package, DataMate (Verity, Applied Cytometry Systems). The CDK1 and CDK4 cell cycle distributions were determined by bi-variate analysis using the Multiple Document Interface WIN MDI (Shareware).

The results are shown in FIG. 10. Panel A shows the level of CDK4 protein in the uninduced culture at different stages of the cell cycle. It can be seen that most cells are in G1 phase. Panel A also shows that CDK4 protein is expressed in all phases of the cell cycle viz G1, S, G2/M. Panel B shows the same data for the induced culture. It can be seen that the CDK4 protein content of all cells is higher irrespective of which phase of the cell cycle they occupy. There is, however no change in the relative distributions of cells in each cell cycle phase. Panels C and D show similar data for CDK1 protein. CDK1 protein is elevated in response to induction of CDK4 in G1, S and G2/M cell cycle phases. The relative distribution of the cells in each cell cycle phase is similar in the uninduced and induced cultures.

Experiment 7—Determination of the Phosphorylation Status of Rb Family Members in Uninduced and Induced Cultures of 2780 1D/275

Rb p105 is the hypophosphorylated form of pRb which binds E2F/DP transcription factors. Rb p110 is the hyperphosphorylated form of pRb. pRb is hyperphosphorylated by CDK4 protein in normal cells and releases free E2F/DP to initiate transcription of a wide range of genes required for cell division.

The level of Rb p110 and Rb p105 present in uninduced and ponasterone induced 2780 1D/275 at various times after transfection was determined using SDS PAGE and Western blotting. At various time points after transfection, the transfected cells were harvested using Trypsin/Versene (Sigma). The harvested cells were centrifuged at 250G for 5 min. The supernatant was decanted and the pellet was resuspended in PBS and the centrifugation was repeated. The cells were then lysed at 3×10⁷ cells per ml using a denaturing lysis buffer. The denaturing buffer was prepared by dissolving one tablet of complete Mini™ (Roche), a protease inhibitor cocktail, in 10 ml of a solution of 1% w/v SDS, 0.8% v/v Glycerol, 0.05M Tris (pH 6.8). The resultant lysate was then sonicated on ice for 10 seconds, and this solution was then centrifuged at 17,500G for 30 minutes at 4° C. The supernatant was then aliquoted, and stored at −80° C.

The protein content of each lysate was determined using a Micro-BSA Protein Determination Kit (Pierce), and the lysates were run on a discontinuous gel of 4%T, 2.6%C stacking gel, with a 10%T, 2.6%C resolving gel using the Lammelli system.

The resolved proteins were then transferred to a nitrocellulose membrane (Protran, Schleicher & Schuell) by electroblotting. Following electrophoretic transfer the membranes were ‘blocked’ for 1 hour with 10%w/v Marvel in TBS at room temperature. The membranes were then probed using a two-stage antibody detection method. The first stage used antibodies generated to human p110 (Mouse monoclonal antibody IF8, Santa Cruz Biotechnology) and p105 (Mouse monoclonal antibody NCL-RB_{—}358, Novacastra). The second stage involved the subsequent probing of the membrane using Horse Radish Peroxidase conjugated Sheep anti-mouse IgG (Serotec, cat. no. AAC10P). The protein bands were then detected using Luminol based Chemoluminescent reagent ECL™ Western blotting detection reagent Kit (from Amersham pharmacia biotech, cat. no. RPN2106), and Hyperfilm™ MP (from Amersham pharmacia biotech, cat. no. RPN1675K). The protein band signals obtained following probing of the membranes as described above were then analysed using the Phoretix software system (Phoretix 1D Gel Analysis, from Non Linear Dynamics Ltd).

Panel A of FIG. 11 shows that a low level of hyperphosphorylated p110 is present at all time points in transfected cells which have not been ponasterone stimulated. Panel A also shows that there is no increase in the levels of hyperphosphorylated pRb (p110) phosphorylation with time following ponasterone induction of CDK4 expression. Instead, hyperphosphorylated pRb (p110) is virtually undetectable in induced cells. Panel B of FIG. 11 shows that in uninduced cells, most CDK4 is present in the phosphorylated (p110) form. However, 24 h and 48 h after ponasterone induction, hypophosphorylated p105 becomes apparent in transfected cells.

Experiment 8—Determination of Metastasis Ability RAMA37 Cells Transfected with Human CDK4 DNA.

RAMA37 cells (derived from immortalised normal rat mammary epithelial cells) were transfected with a mammalian expression construct comprising the human CDK4 ORF in pcDNA3. As controls, pcDNA3 vector alone and pcDNA3 containing the human p53 ORF were transfected into RAMA37 cells.

Transfectants were selected with neomycin and then cultured. The transfected cell lines were tested for mRNA and protein expression by Northern and Western Blotting respectively to detect transfectants expressing increased levels of the transfected genes. The high expressing cells lines were grown up in tissue culture to yield sufficient cells for inoculation into animals.

2×10⁶ cells of each transfected cell line were inoculated into a single site subcutaneously into each of 20 female Furth Wistar rats. The rats were sacrificed and metastasis was detected by removal and macroscopic inspection of the rat lungs. The results are shown in Table 1.

TABLE 1

| Transfectant cell line | Tumours at inoculation site | Metastasis |
|---|---|---|
| pcDNA3 | 14/20 | 0/14 |
| p53pcDNA3 | 7/20 | 0/7 |
| CDK4pcDNA3 | 10/20 | 9/10 |

Table 1 clearly shows that RAMA37 cells expressing increased levels of CDK4 give a high incidence of pulmonary metastases.

Experiment 9—Western Blot Showing Changes in p9Ka Expression Following Induction of CDK4 Expression.

The level of CDK4 protein and p9Ka protein present in uninduced and induced 2780 1D/275 generated was determined at 24, 40 and 48 h following induction of CDK4 expression with ponasterone or mock induction. At 20, 40 and 48 h after induction/mock induction, the induced cells were harvested using Trypsin/Versene (Sigma). The harvested cells were centrifuged at 250G for 5 min. The supernatant was decanted and the pellet was resuspended in PBS and the centrifugation was repeated. The cells were then lysed at 3×10⁷ cells per ml using a denaturing lysis buffer. The denaturing buffer was prepared by dissolving one tablet of complete Mini™ (Roche), a protease inhibitor cocktail, in 10 ml of a solution of 1% w/v SDS, 0.8% v/v Glycerol, 0.05M Tris (pH 6.8). The resultant lysate was then sonicated on ice for 10 seconds, and this solution was then centrifuged at 17,500G for 30 minutes at 4° C. The supernatant was then aliquoted, and stored at −80° C.

The protein content of each lysate was determined using a Micro-BSA Protein Determination Kit (Pierce), and the lysates were run on a discontinuous gel of 4%T, 2.6%C stacking gel, with a 10%T, 2.6%C resolving gel using the Lammelli system. Prestained markers were run on either side of the samples

The resolved proteins were then transferred to a nitrocellulose membrane (Protran, Schleicher & Schuell) by electroblotting. Following electrophoretic transfer the membrane was cut across the 27 kilodalton molecular weight marker. The membranes were ‘blocked’ for 1 hour with 10%w/v Marvel in TBS at room temperature. The upper portion of the membrane was probed using a two-stage antibody detection method. The first stage involved probing the membrane with a 1 in 250 dilution of an antibody raised against human CDK4 (Santa Cruz, cat. no. Sc-260) for 1 h at room temperature. The second stage involved the subsequent probing of the membrane using a 1 in 1000 dilution of Horse Radish Peroxidase conjugated Sheep anti-rabbit IgG (Serotec, cat. no. STAR54). The protein bands were then detected using Luminol based Chemoluminescent reagent ECL™ Western blotting detection reagent Kit (from Amersham pharmacia biotech, cat. no. RPN2106), and Hyperfilm™ MP (from Amersham pharmacia biotech, cat. no. RPN1675K).

The lower portion of the membrane was probed with a 1 in 1000 dilution of rabbit anti-human p9ka polyclonal antibody (DAKO A5114) for 16 hours at room temperature. The second stage involved the subsequent probing of the membrane using a 1 in 1000 dilution of Horse Radish Peroxidase conjugated Sheep anti-rabbit IgG (Serotec, cat. no. STAR54). The protein bands were then detected using Luminol based Chemoluminescent reagent ECL™ Western blotting detection reagent Kit (from Amersham pharmacia biotech, cat. no. RPN2106), and Hyperfilm™ MP (from Amersham pharmacia biotech, cat. no. RPN1675K).

The results are presented in FIG. 12. Panel A shows that p9ka protein is undetectable at 20, 40 and 48 following mock induction. CDK4 protein is present in all uninduced samples in an approximately equal amount. The amount of CDK4 protein in each sample of panel B is greater than the level in the uninduced cell lysates. In addition, the level of CDK4 protein increases with induction time. Small amounts of p9Ka protein are present. These also increase with longer induction times. Without being bound by theory, the applicant hypothesises that the CDK4 gene product elevates the level of the p9Ka gene product.

The invention will be described further with the aid of Examples. These examples illustrate the scope of the invention but are not intended to be limiting.

### EXAMPLE 1

### Identification of p27^{KIP1} Protein as a Critical Normal Gene Product

p27^{KIP1} mutations have not been found, or are extremely rare in various cancers (Sgambato et al. (2000) J. Cell Physiol. 183: 18-27). In addition, the protein product of this gene is paradoxically upregulated in human mammary cancer cell lines and primary human cancers of the breast (Weinstein (2000) Carcinogenesis 21: 857-864). Upregulation has also been reported in a subset of human cancers including oesophagus, breast, colon and small cell lung cancers (Fredersdorf et al. (1997) Proc Natl. Acad. Sci 94: 6380-6385; Yatabe et al. (1998) Cancer Res. 58: 1042-1047). Whereas p27^{KIP1} protein levels fluctuate throughout the cell cycle in normal mammary epithelium, they remain high throughout the cell cycle in breast cancer cell lines (Sgambato et al. (1997) Clin Cancer Res. 3: 1879-1887). This implies that p27^{KIP1} protein functions as a critical normal gene product in some breast, colon, oesophagus and small cell lung cancers. Therefore, chemotherapeutic agents that target the p27^{KIP1} gene product are likely to prove suitable chemotherapeutic agents for the treatment of these cancers.

This is unexpected as p27^{KIP1} protein functions as an inhibitor of cell division in normal cells. Before the realisation that p27^{KIP1} protein was a critical normal gene product, it would not have been obvious to treat cancer by disrupting an inhibitor of cell division. The applicant considers that p27^{KIP1} may act as a critical normal gene by promoting apoptosis. Increasing expression of p27^{KIP1} protein by retroviral transduction (Banerji et al. (2001) Oncogene 20: 7352-7367; Li et al. (2000) World J. Gastroenterol. 6: 513-521) or transfection of zinc inducible p27 causes GI cell cycle arrest and subsequent apoptosis. p27^{KIP1} induced apoptosis has been shown to be associated with inhibition of CDK2 complexed to cyclin E (Masuda et al. (2001) Am. J. Pathol. 158: 87-96) or cyclin A (Hiromura et al. (1999) J. Clin. Invest. 103: 597-604). p27^{KIP1} protein may, however, function as a critical normal gene product by an alternative mechanism to that of causing apoptosis as high levels of p27^{KIP1} protein has been reported to be related to poor prognosis but not an increased apoptotic index in colorectal adenocarcinomas (Tenjo et al., (2000) Oncology 58: 45-51).

As discussed above, the applicant hypothesises that in clinical cancers there is a state of chaos where the normal complex mechanistic controls of cell division and death no longer operate. Instead, the cellular fate is determined by the non-stochastic balance of expression of genes promoting cellular proliferation with those promoting cell death. This cell death may be by apoptosis as has been reported to occur as a result of E2F overexpression but could also take the form of necrosis. Cell proliferation and apoptosis may both be consequences of E2F overexpression. For tumours to survive and proliferate there would be expected to be controlling gene products maintaining E2F at levels which promoted cell division but not apoptosis. Exerting such control is suggested to be a potential function of Critical Normal Gene Products. Without being bound by theory, the applicant postulates that p27^{KIP1} protein is involved in controlling the level of E2F.

p27^{KIP1} protein does not act as a critical normal gene product in all cancers. Many aggressive, poorly differentiated tumours (including certain lymphomas, gliomas, small cell lung cancers and cancers of the breast, stomach, colon, prostate and oral cavity) display reduced expression of p27^{KIP1} protein. Tumours for which anti-p27^{KIP1} treatment would be suitable may be identified by a two step test. The first step involves measuring the level of p27^{KIP1} mRNA or p27^{KIP1} protein in a sample of cancer cells or an extract therefrom, and in a control sample as described above. The level of mRNA or protein in the cancer sample must be greater than or equal to that in the control sample for anti-p27^{KIP1} treatment to be suitable. The second step is to identify the sequence of p27^{KIP1} in both tumour and control samples. This can be done by either sequencing the gene, or by the use of antibodies specific to the wild type protein. For anti-p27^{KIP1} treatment to be suitable, the gene from the cancer sample must be wild type (i.e. have the same sequence as the gene from the control sample) or contain no mutations that affect the critical functioning of the protein. Antisense cDNA to p27^{KIP1} mRNA or direct inhibitors of the p27^{KIP1} protein are likely to be effective chemotherapeutic agents for treatment of tumours in which p27^{KIP1} is a critical normal gene product.

### EXAMPLE 2

### Identification of Chemotherapeutic Agents for Cancers in Which Retinoblastoma is a Critical Normal Gene

Retinoblastoma has been reported to be elevated in human leukaemias and breast colon and bladder cancers (Weinstein (2000) Carcinogenesis 21: 857-864; Wildrick and Boman (1994) Mol Carcinogenesis 10: 1-7). Furthermore, a progressive increase in the expression of pRb protein has been found during the multistage process of colon carcinogenesis (Yamamoto et al. (1999) Clin Cancer Research 5: 1805-1815). Thus, pRb is a critical normal gene product in such cells.

The applicant postulates that pRb acts as a Critical Normal Gene Product in cancer cells in which it is upregulated by inhibiting apoptosis. Apoptosis occurs as a consequence of overexpressed E2F-1 in the full malignant phenotype. Through its interaction with E2F, pRb may inhibit the apoptotic pathway (Harbour and Dean, (2000) Nature Cell Biol. 2: E65-67). Lack of pRb protein has been shown to correlate with increased sensitivity to UV radiation induced apoptosis in human breast cancer cells (Carlson and Ethier (2000) Radiat. Res. 154: 590-599) whilst transduction with pRb containing adenovirus Ad-Rb attenuates p53 induced apoptosis in cervical cancer cell lines (Ip et al., (2001) Eur. J. Cancer 37: 2475-2483). These studies support the hypothesis that pRb acts as a Critical Normal Gene Product by protecting cancer cells from apoptosis.

| | |
|---|---|
| 5′-GTCATGCCGCCCAAAACC-3′: | SEQ1 |
| 5′-GGTTTTGGGCGGCATGAC-3′: | SEQ2 |

Sense and antisense phosphorothionate oligonucleotide with the sequence set out in SEQ1 and SEQ2 respectively were synthesised in a 380B DNA synthesiser. Each oligonucleotide was premixed with lipofectin reagent and diluted in DMEM (Gibco). 6 cm plates of HCT116 cells at 40-50% confluence were rinsed once with 4 ml serum free DMEM and transfected with the oligonucleotides at a final concentration of 1 μM and the lipofectin reagent at a final concentration of 19 μg/ml according to the manufacturer's instructions (Life Technologies Inc., Gaithersburg, Md.). After 4 hours, Foetal Calf Serum (Gibco) was added to a final concentration of 10%. A lipofectin only transfection was performed as a control. Cell extracts were harvested after 48 h and examined by Western blot analysis with a pRb antibody. Equal loading of protein samples was confirmed by Coomassie blue staining or by immunoreactivity with an anti-actin antibody (Sigma). The intensities of the Rb bands were quantised with an image scanner (Molecular Dynamics). The results show that treatment of the cells with the antisense oligonucleotide reduced the level of pRb expression to about 30% of the control culture treated only with lipofectin. The treatment with the sense oligonucleotide led to about a 5% reduction in the level of pRb. Growth curves indicated that the culture treated with the antisense oligonucleotide displayed growth inhibition when compared with the lipofectin-treated control culture; but that treatment with the sense oligonucleotide had no significant effect on growth. Apoptosis in the cultures 48 h after transfection was tested for using the TUNEL assay. An in situ apoptosis detection kit ApopTag (Oncor, Gaithersburg, Md.) was used as recommended by the manufacturer. The percentage of TUNEL positive cells was 1.5+/−1% for the lipofectin treated culture, 3+/−0.5% for the culture transfected with the sense oligonucleotide and 18+/−0.5% for the culture transfected with the antisense oligonucleotide. Thus, the sense oligonucleotide caused about a two-fold increase in apoptosis, but the antisense oligonucleotide caused over a 10-fold increase in apoptosis. As the antisense oligonucleotide inhibits growth and increases apoptosis, it is thus an effective chemotherapeutic agent.

The retinoblastoma gene is not a ubiquitous Critical Normal Gene as it is inactivated in a large number of human cancers. Tumours for which anti-Rb treatment would be suitable may be identified by a two step test. The first step involves measuring the level of Rb in a sample of cancer cells or an extract therefrom, and in a control sample. This can be achieved by measuring levels of Rb protein or mRNA as described above. The level of protein in the cancer sample must be greater than or equal to that in the control sample for anti-Rb treatment to be suitable. The second step is to identify the sequence of Rb in both tumour and control samples. This can be done by either sequencing the gene, or by the use of antibodies specific to the wild type protein. For anti-Rb treatment to be suitable, the gene from the cancer sample must be wild type (i.e. have the same sequence as the gene from the control sample) or contain no mutations that affect the critical functioning of the protein. For such tumours, the antisense agent with the sequence set out in SEQ2 is likely to be an effective chemotherapeutic agent.

### EXAMPLE 3

### Identification of p9Ka Protein as a Critical Normal Gene Product for Metastasis

3 μm tissue sections were cut from archival formalin-fixed paraffin embedded specimens from 287 patients who had presented with primary operable breast cancer between the years 1976 to 1982 in the Merseyside Region as reported previously (Winstanley et al. Br. J. Cancer (1991) 63: 447-450). The tissue sections were dewaxed (Warburton et al. J. Histochem. Cytochem. (1982) 30: 667-676) endogenous peroxidase activity was blocked with H₂O₂ (Streefkerk J. Histochem. Cytochem (1972) 20: 829-831), and the p9Ka antigen was visualised by the ABC method (Hsu et al. J. Histochem Cytochem (1981) 29: 577-580) using horseradish peroxidase and diethylaminobenzene to produce a brown colouration on the histological sections. The cell nuclei were counterstained blue by Meyers Haemalum (Warburton et al. J. Histochem. Cytochem. (1982) 30: 667-676). The percentage of carcinoma cells stained for p9Ka protein was determined from two sections of each specimen, 5 to 10 fields per section (250× magnification; about 200-400 cells) in a microscope by two independent observers. In sections from 107 patients, <1% of the carcinoma cells were stained for p9Ka protein, in sections from 45 patients, 1-5% of the carcinoma cells were stained for p9Ka protein, and in 135 patients, >5% of the carcinoma cells were stained for p9Ka protein.

Therefore, in a large number of primary breast cancers, p9Ka protein is expressed or overexpressed by the cancer cells. Overexpression of p9Ka has been suggested to be associated with metastatic spread to local lymph nodes in human breast cancer (Albertazzi et al. DNA Cell Biol. (1998) 17: 335-342), and shown to be present in liver metastases arising from colon carcinomas (Takenaga et al. Clin Cancer Res. (1997) 3: 2309-2316). This implies that p9Ka protein must be present in cells at normal levels or higher for metastasis to occur. In support of this hypothesis, p9Ka protein has been shown to induce metastatic capability in non-metastatic rat and mouse mammary tumour cells (Davies et al. Oncogene (1993) 8: 999-1008; Grigorian et al. Int. J. Cancer (1996) 67: 831-841) and to co-operate with oncogenic changes in non-metastatic tumour cells to yield metastatic tumours (Ambartsumian et al. Oncogene (1996) 13: 1621-1630; Davies et al. Oncogene (1996) 13: 1631-1637).

Anti-p9Ka treatment may prevent tumours expressing p9Ka metastasising. As the process of metastasis is responsible for the majority of deaths arising from cancers of solid tissues, treatment with anti-p9Ka may reduce mortality. Tumours for which anti-p9Ka treatment would be suitable may be identified by a two step test. The first step involves measuring the level of p9Ka mRNA or p9Ka protein in a sample of cancer cells or an extract therefrom, and in a control sample as described above. The level of mRNA or protein in the cancer sample must be greater than or equal to that in the control sample for anti-p9Ka treatment to be suitable. The second step is to identify the sequence of p9Ka in both tumour and control samples. This can be done by either sequencing the gene, or by the use of antibodies specific to the wild type protein. For anti-p9KA treatment to be suitable, the gene from the cancer sample must be wild type (i.e. have the same sequence as the gene from the control sample) or contain no mutations that affect the critical function of the protein. Antisense cDNA to p9KA mRNA or direct inhibitors of the p9Ka protein are likely to be effective chemotherapeutic agents for treatment of tumours in which p9Ka is a critical normal gene product.

### EXAMPLE 4

### Identification of CDK1 and CDK4 as Ubiquitous Critical Normal Genes

CDK1 and CDK4 are two proteins that have recently been found to be consistently co-elevated in a wide range of human cancer cell lines (Seabra and Warenius (1998) Proc Am Ass Cancer Research 39: 442). CDK1/CDK4 protein levels in every cell line were greater than those in normal fibroblast or keratinocytes. Similar co-elevation of these proteins has also been observed in clinical samples of human colon and breast cancers as compared to adjacent normal tissue. The CDK4/CDK1 relationship found in human cancer cells (Warenius (2002) Anticancer Res. In press) is not present in normal keratinocytes and fibroblasts (FIGS. 3 and 4). Also, whereas there is a highly significant correlation between the levels of the CDK1 and CDK4 proteins in cancer cells, particularly in those bearing p53 mutations, no correlation is seen in primary cultures of human fibroblast or keratinocytes from different individuals (FIGS. 3 and 4).

No mutations have been recorded in CDK1 in the literature to date and the only mutation that has been recorded in CDK4 is rare and involves the p16^{INK4} binding regions of the protein (Sotillo et al, (2001) EMBO J. 20: 6637-47); Rane et al (2002) Mol. Cell. Biol. 22: 644-56) which would make CDK4 refactory to p16^{INK4} inhibition but not necessarily disrupt any other CDK4 functions. In fact, transgenic mice engineered to carry this mutation (replacement of Arg 24 by Cys) have embryonic fibroblasts which show escape from cellular senescence and increased tumorigenesis. Thus, this mutation does not appear to prevent a critical normal function of CDK4.

Thus, CDK1 and CDK4 meet the criteria defining a critical normal gene. They are overexpressed in cancer cells and remain wild type or do not have any mutations that affect the critical normal function of the protein. Cancer cells which do not overexpress these gene products in a ratio of 0.6 to 1.6 undergo cell death. In support of this assertion, FIG. 8 shows that dying L23COR cells exhibit a CDK1/CDK4 ratio of 0.3 whilst proliferating L23COR cells have a CDK1/CDK4 ratio of 1.0. Therefore, it appears that if the critical normal function of CDK1 and CDK4 is removed from cancer cells, the cancer cells undergo cell death. This is evidence that these gene products perform a critical normal function within human cancer cells. As a large number of cancers show co-elevation of CDK1 and CDK4 proteins, the CDK1 and CDK4 gene products may be more ubiquitous targets for novel anticancer drug development. Antisense oligonucleotides complementary to CDK1 or CDK4 mRNA may thus be effective chemotherapeutic agents.

The critical normal function of the CDK1 gene product may be to prevent apoptosis and thereby promote tumour cell survival. Increased expression of CDK1 and a significantly decreased apoptotic index have both been detected in recurrent as compared to primary colonic tumours (Seong et al., (1999) Int. J. Radiat. Biol. Phys 45: 1167-73). Moreover, inhibition of CDK1 protein with olomucine and roscovitine results in increased levels of apoptosis (Schutte et al., (1997) Exp. Cell Res. 236: 4-15). Without being bound by theory, the applicant considers that CDK1 protein may regulate apoptosis through phosphorylation of survivin at mitotis. Phosphorylation of survivin prevents dissociation of survivin-caspase 9 complex on the mitotic apparatus and thus prevents the release of caspase 9 which promotes apoptosis (O'Conner et al., (2000) Proc. Natl. Acad. Sci., USA 97: 13103-13107). The region of CDK1 that interacts with survivin is likely to be a good target for chemotherapeutic agents.

The applicant has identified a critical normal function of CDK4 in cancer cells. It appears that CDK4 acts to elevate CDK1, p9Ka and possibly CDK2, CDK6 and p27 by a mechanism that is independent of its role in the cell cycle. The region of CDK4 protein that mediates this function is unknown. CDK4 protein has many known functional regions with regard to its action in normal cells (see FIG. 3). These involve regions responsible for binding to Cyclin D, p16^{INK4}, p27^{KIP1}, p21^{WAF1/CIP1} and pRb. There are also regions carrying out CDK kinase functions. These include the CDK4 site which phosphorylates pRb, Tyrosine 17 whose phosphorylation/dephosphorylation by kinases and phosphatases controls CDK4 kinase activity, and Threonine 164 whose phosphorylation is required for the activation of CDK4 kinase. The novel function of CDK4 in clinical cancer cells described here does not involve the known functions of the regions described above. The applicant suggests, without being bound by theory, that the relatively long length of peptide between amino acids 172 and 285 of the CDK4 protein, to which no function has yet been ascribed, contains further functional regions which may be involved in the mechanism by which CDK4 causes elevation of CDK1, p9Ka, hypophosphorylated pRb, CDK2, p27KIP1 and possibly other Critical Normal Gene Products in Human Clinical Cancer cells. Inhibitors to this region of the protein are likely to be effective agents for the treatment of cancer. Antisense agents complementary to the region of the CDK4 mRNA encoding amino acids 172-285 are likely to be effective agents for the treatment of cancer.

The experimental data presented in this application suggests that the critical normal function of the CDK4 gene product resides in its ability to elevate the levels of other critical normal genes which are crucial for the survival of cancer cells and metastasis.

The CDK4 gene product is known to bind to p9Ka. The applicant hypothesises that CDK4 and 9Ka may, in combination, promote metastasis. In support of this, RAMA 37 cells (derived from a rat mammary cell line) transfected with either p9Ka DNA or with CDK4 DNA are able to metastasise in an assay for metastasis whilst untransfected cells do not metastasise (see Table 1).

### EXAMPLE 5

### Identification of Telomerase as a Ubiquitous Critical Normal Gene

Telomerase is silent in most adult normal tissues (except stem cells), but is re-expressed in all tumours where it is required to overcome the telomere shortening which accompanies each cell division (Preston (1997) Rad. Research 147: 529-534). It therefore acts as a Critical Normal Gene Product. Telomere shortening ultimately leads to cancer cell death. Telomerase inhibitors remove the capacity of cancer cells to regenerate telomeres and thus to undergo unlimited proliferation. The efficacy of telomerase inhibitors would be expected however to be dependent upon telomere length at the time treatment is implemented. Advanced tumours with long telomeres and within only a few doublings of the tumour load required to kill the patient would be less likely to be arrested by anti-telomerase treatment before they caused death than early tumours which would have undergo may more doublings and telomere shortenings to produce the same outcome. Thus whilst telomerase potentially provides a ubiquitous Critical Normal Gene Product target in terms of gene expression, therapeutic strategies directed at this target may not be effective in all tumours.

1. A method of screening for an agent effective in the treatment of a cancer, which method comprises:
a) selecting a putative agent that is likely to disrupt a critical normal gene product present in said cancer;
b) determining the cytotoxic effect of, and/or the growth inhibiting effect of the putative agent on a cancer cell sample; and
c) identifying an effective agent as an agent which is cytotoxic to, and/or inhibiting to the growth of the cancer cell sample.

2. A method according to claim 1, further comprising determining the cytotoxic effect of, and/or the growth inhibiting effect of the putative agent on a control cell sample and identifying an effective agent as an agent which is more cytotoxic to, and/or more inhibiting to the growth of the cancer cell sample than the control cell sample.

3. The method according to claim 1 or claim 2, wherein the cancer cell sample and/or the control cell sample are extracted from a subject.

4. The method according to claim 1 or claim 2, wherein the cancer cell sample and/or the control cell sample are derived from a cell line.

5. The method according to claim 4, wherein the cell line is transfected with a critical normal gene.

6. The method according to any preceding claim, wherein the disruption of the critical normal gene product inhibits a critical normal function of that gene product.

7. The method according to claim 6, wherein the critical normal gene product has a plurality of functions and disruption inhibits one or more functions of the gene product critical to successful division and continued cell survival.

8. The method according to any preceding claim, wherein the critical normal gene is one or more of p27^{KIP1}, Rb, CDK1, CDK4, and telomerase.

9. The method according to claim 8, wherein the cancer cell sample consists of one or more cells in which the CDK1 and CDK4 gene products are co-elevated, and wherein an effective agent further disrupts the co-elevation of the CDK1 and CDK4 gene products in the cancer cell sample.

10. The method according to claim 9, wherein the ratio of the levels of the CDK1/CDK4 gene products in the cancer cell sample is in the range of 0.6 to 1.6.

11. The method according to claim 9 or claim 10, wherein the control cell sample consists of one or more cells in which the CDK1 and CDK4 gene products are not co-elevated and wherein an effective agent is more cytotoxic to, and/or more inhibiting to the growth of the cancer cell sample than the control cell sample.

12. The method according to any preceding claim, which further comprises testing the safety of the effective agent in an animal model system.

13. A method according to any preceding claim, wherein said cancer is breast, prostate, colon, bladder, stomach, pancreatic or oesophagus cancer, small cell lung cancer, non-small cell lung cancer, malignant melanoma, neuroblastoma or a leukaemia.

14. A method of screening for an agent effective in preventing a cancer from undergoing metastasis, which method comprises:
a) selecting a putative agent that is likely to disrupt a critical normal gene product present in said cancer;
b) determining the ability of a sample of metastatic cancer cells to undergo metastasis in the presence of said agent; and
c) identifying an effective agent as an agent which, when present, reduces the ability of said sample of metastatic cancer cells to metastasise;
wherein said putative agent is a peptide or protein.

15. A method according to claim 14, wherein said critical normal gene for metastasis is p9Ka, CDK4 or osteopontin.

16. An agent for use in medicine, which agent is capable of disrupting a critical normal gene product in such a manner as to be cytotoxic to, or to inhibit the growth of a cancer cell.

17. An agent according to claim 16, wherein said agent is more cytotoxic to, or more inhibiting to the growth of, a cancer cell sample than a control cell sample.

18. An agent according to claim 16 or claim 17, wherein said critical normal gene is p27^{KIP1}, Rb, CDK1, CDK4, or telomerase.

19. An agent according to any of claims 16 to 18, wherein said agent is an antisense oligonucleotide.

20. An agent according to claim 19, wherein said antisense agent has the sequence set out in SEQ2.

21. An agent according to claim 17, wherein said antisense agent is complementary to the region of the CDK4 mRNA encoding amino acids 172-285.

22. An agent according to any of claims 16 to 18, wherein the cancer cell sample consists of one or more cells in which the CDK1 and CDK4 gene products are co-elevated, and wherein said agent further disrupts the co-elevation of the CDK1 and CDK4 gene products in the cancer cell sample.

23. An agent according to claim 22, wherein the ratio of the levels of the CDK1/CDK4 gene products in the cancer cell sample is in the range of 0.6 to 1.6.

24. An agent according to claim 22 or claim 23, wherein the control cell sample consists of one or more cells in which the CDK1 and CDK4 gene products are not co-elevated and wherein an effective agent is more cytotoxic to, and/or more inhibiting to the growth of a cancer cell than a control cell.

25. An agent for use in medicine, which agent is capable of disrupting a critical normal gene product in such a manner as to reduce the ability of a metastatic cancer cell to metastasise.

26. An agent according to claim 25, wherein said critical normal gene is p9Ka, CDK4 or osteopontin.

27. A pharmaceutical composition comprising the agent according to any one of claims 16 to 26, and/or a diluent or excipient.

28. A pharmaceutical composition according to claim 27, which is suitable for parenteral administration.

29. A method of manufacturing a pharmaceutical composition as defined in claim 27 or claim 28, which method comprises identifying an effective agent according to the screening method defined in any of claims 1 to 15, and manufacturing a pharmaceutical composition comprising said effective agent.

30. Use of an agent which agent is capable of disrupting a critical normal gene in such a manner as to be cytotoxic to, or inhibiting to the growth of, a cancer cell, in the manufacture of a medicament for the treatment of cancer.

31. Use according to claim 30, wherein the agent is more cytotoxic to, and/or more inhibiting to the growth of a cancer cell than a control cell.

32. Use according to claim 30 or claim 31, wherein the agent is an agent as defined in any one of claims 17 to 24.

33. Use of an agent which agent is capable of reducing the ability of a metastatic cancer cell to metastasise in the manufacture of a medicament for the treatment of cancer.

34. Use according to claim 33, wherein the agent is an agent as defined in claim 25 or claim 26

35. Use according to any of claims 30 to 34, wherein said cancer is breast, prostate, colon, bladder, stomach, pancreatic or oesophagus cancer, small cell lung cancer, non-small cell lung cancer, malignant melanoma, neuroblastoma or a leukaemia.

36. A method of treating a patient having cancer comprising:
a) identifying a critical normal gene present in said cancer;
b) treating the patient with an agent or pharmaceutical composition capable of disrupting said critical normal gene product, as defined in any of claims 16 to 28.

37. A method of treating a patient having cancer according to claim 36 further comprising identifying one or more additional critical normal genes present in said cancer and further treating the patient with one or more additional agents or pharmaceutical compositions capable of disrupting these additional critical normal genes as defined in any of claims 16 to 28.

38. A method according to claim 36 or claim 37, wherein the identification of critical normal genes takes place by Western blotting, FACS analysis, or by hybridization of an oligonucleotide probe to the sample.

39. A method of selecting a treatment for a patient having a cancer, which method comprises:
a) identifying a critical normal gene present in said cancer;
b) selecting an agent for treatment which agent disrupts said critical normal gene product and is an agent as defined in any of claims 16-28.

40. A method of selecting a treatment according to claim 39, wherein the cancer contains co-elevated levels of the CDK1 and CDK4 gene products, and wherein the agent selected for treating the patient disrupts the co-elevation of the CDK1 and CDK4 gene products.

41. The method according to claim 40, wherein the ratio of the levels of the CDK1/CDK4 gene products in the cancer is in the range of 0.6 to 1.6.

42. A method of selecting a treatment according to any of claims 39 to 41 which further comprises identifying one or more additional critical normal genes present in said cancer, and selecting one or more additional agents or pharmaceutical compositions capable of disrupting these additional critical normal genes as defined in any of claims 16 to 28 for treating said patient.

43. A kit for selecting and providing a suitable treatment for a patient having a cancer comprising:
a) a means for identifying a critical normal gene present in said cancer; and
b) an agent capable of disrupting a critical normal gene as defined in any of claims 16 to 28.

44. A kit according to claim 43, wherein said means for identifying a critical normal gene is an antibody recognising the product of said critical normal gene.

45. A kit according to claim 43 or claim 44, wherein said critical normal gene is p27^{KIP1}, Rb, CDK1, CDK4, p9Ka, osteopontin or telomerase.

46. A method for identifying critical normal genes, which method comprises:
a) detection of a gene product in L23COR cells that are quiescent or proliferating;
b) detection of said gene product in dying L23COR cells; and
c) identifying a critical normal gene as a gene product which is present at higher levels in quiescent or proliferating L23COR cells than in dying L23COR cells.

47. A method according to claim 46, wherein the method of detection of said gene product takes place by FACS analysis, immunocytochemistry, Western Blotting or Northern Blotting.


SEQUENCE LISTING (4 sequences)

SEQ ID NO: 1 - DNA, 18 bp, Artificial
  Sense phosphorothionate oligonucleotide of E2F
gtcatgccgcccaaaacc

SEQ ID NO: 2 - DNA, 18 bp, Artificial
  Antisense phosphorothionate oligonucleotide of E2F
ggttttgggcggcatgac

SEQ ID NO: 3 - DNA, 32 bp, Artificial
  PCR primer
cccgaattcagaatggtcacctctcgatatga

SEQ ID NO: 4 - DNA, 31 bp, Artificial
  PCR primer
cccgcggccgctgctcactctggattacctt